(19) United States
(12) Patent Application Publication
(10) Pub. No.: US 2002/0055172 A1
(43) Pub. Date: May 9, 2002

(54) Multiple promoter expression constructs and methods of use

(76) Inventor: John J. Harrington, Mentor, OH (US)

Correspondence Address: SHANKS & HERBERT, TransPotomac Plaza, Suite 306, 1033 N. Fairfax St., Alexandria, VA, 22314, US

(21) Appl. No.: 09/729,416

(22) Filed: Dec. 5, 2000

Related U.S. Application Data
(63) Continuation of application No. 09/414,369, filed on Oct. 7, 1999, now abandoned.

(51) Int. Cl. C12N 15/867; C12N 15/63; C07H 21/04

(52) U.S. Cl. 435/456; 435/473; 435/320.1; 536/23.2

(57) ABSTRACT

The invention is directed to improved methods for gene expression using vectors with multiple promoters. Multiple promoters are used in nucleic acid constructs to provide increased expression of a desired nucleic acid sequence. The sequence is introduced into a vector by conventional cloning or is expressed from an endogenous sequence in the genome that is activated by the vector containing the multiple promoters.

### FIELD OF THE INVENTION

The invention is directed to improved methods for gene expression using vectors with multiple promoters. Multiple promoters are used in nucleic acid constructs to provide increased expression of a desired nucleic acid sequence. The sequence is introduced into a vector by conventional cloning or is expressed from an endogenous sequence in the genome that is activated by the vector containing the multiple promoters.

### BACKGROUND OF THE INVENTION

Over-expression of genes is an important step toward developing new therapeutics and diagnostics. By over-expressing a gene of interest, large amounts of protein can be produced for subsequent analysis and testing. Likewise, once a gene product is deemed to have commercial value, over-expression is necessary to produce material for commercialization. Using current methods, gene over-expression and protein production is a time-consuming and expressive process.

Current methods of protein expression involve placing a promoter element in operable linkage with a gene of interest to produce an expression cassette. For purposes of this document, an expression cassette is any polynucleotide sequence containing a promoter operably linked to a gene of interest. Methods of linking promoter elements to genes include cloning and ligation in vitro (1), homologous recombination in situ (2-6), and non-homologous recombination in vitro or in situ (7). Variations of these approaches have also been described.

Regardless of the method used for linking the promoter element to a gene of interest, the expression cassette, if not already present in a suitable host cell, is introduced into a host cell to allow the promoter element to express the gene of interest. Once in the cell, the gene of interest will be transcribed and translated to produce a protein of interest.

Unfortunately, expression levels in cells containing the expression cassette are often too low for many purposes. In order to achieve higher levels of gene expression, typically the copy number of the expression cassette needs to be increased. By increasing the number of copies of the gene of interest, the cell's expression machinery has a larger number of templates to act upon. As a result, more mRNA is produced, which in turn, leads to increased protein production.

A variety of methods for increasing expression cassette copy number exist.

The use of any one method depends on whether the expression cassette is episomal or integrated into the host cell genome. For example, to increase protein expression from an episomal vector, the copy number of the episome can be increased by introducing higher concentrations of the vector into the cell or by including a viral origin of replication on the expression vector (8-11). Alternatively, to increase protein expression from an integrated expression cassette, an amplifiable marker may be included on the expression cassette. Using the appropriate selection, cells containing increased copies of the expression cassette can be isolated (12). Regardless of the method used, in general, as copy number increases, the protein expression level also increases. Unfortunately, the process of gene amplification is time consuming and expensive.

Thus, current methods of gene expression suffer from a number of problems. For example, in cells containing unamplified conventional expression cassettes, expression levels are too low for many purposes. Furthermore, amplifying copy number is time consuming and expensive, and in some cases, does not result in the desired levels of protein expression. As a result, there exists a need in the art for methods of high level gene expression without gene amplification. There also exists a need for methods capable of increasing protein expression levels in cells containing amplified expression cassettes.

### SUMMARY OF THE INVENTION

The present invention, therefore, is generally directed to vectors and methods for expressing nucleic acid molecules, including eukaryotic genes. Expression can be achieved by conventional cloning, or otherwise introducing a nucleic acid molecule, such as a cDNA molecule or genomic fragment, into the vectors of the invention, followed by introduction into a suitable host cell. Alternatively, the vectors of the invention can be operably linked to an endogenous gene by integrating the vector into the genome of a host cell by homologous, nonhomologous, or site-specific recombination.

The vectors of the invention comprise multiple promoter elements positioned in tandem along a polynucleotide sequence. Each promoter is operably linked to an exonic sequence followed by an unpaired splice donor site to produce a promoter/exon unit (FIG. 1). The vector can contain one or more promoter/exon units. For example, the vector can contain one, two, three, or four promoter/exon units (FIG. 2). Alternatively, the vector can contain 5 or more promoter/exon units. The range is about 2-5, 6-10, 10-20 or more. The exact number of promoter/exon units required for a particular application depends on promoter strength, splicing efficiency, cell type, the environment in which the gene is expressed (e.g. chromosomal or episomal), and the level of expression desired. To identify the appropriate expression level for a particular application, the number of promoter/exon units can be rapidly tested, by routine experimentation, using the methods set forth herein.

To express a gene of interest using the vectors of the invention, the gene is positioned downstream of, and in the same orientation as, the multi-promoter/exon units, i.e., operably linked. The gene can supply a splice acceptor site to allow efficient splicing from each of the upstream splice donor sites. Alternatively, a splice acceptor site can be engineered into the vector or gene of interest (FIG. 3). The presence of the downstream splice acceptor site, in combination with the upstream splice donor sites associated with each promoter/exon unit, allows each promoter to create an RNA transcript, which in turn, is spliced to produce a mature mRNA molecule capable of being translated into the protein of interest (FIG. 4). Since the gene of interest is operably linked to multiple promoter/exon units, rather than to a single promoter/exon unit, higher levels of transcription can be achieved. Furthermore, since each promoter/exon unit is capable of generating a mature mRNA encoding the protein of interest, the higher levels of transcription lead to an increase in protein production. Thus, unlike previous methods that increase gene expression by increasing the copy number of the gene of interest, the present invention increases protein expression by increasing the number of promoter/exon units associated with the gene of interest. As a result, higher levels of protein expression can be achieved without amplifying the copy number of the gene of interest.

The vectors of the invention, however, can also contain amplifiable markers, and therefore, can be used to produce higher levels of protein production in cells containing amplified copies of the gene of interest. Thus, the present invention is also directed to vectors and methods for increasing gene expression of amplified genes.

The present invention is also drawn to methods of creating operable linkages between the vectors of the invention and genes of interest. In this respect, the vectors can be operably linked to a gene of interest using conventional cloning and ligation, non-homologous recombination including transposition and retroviral insertion (in vivo or in situ), homologous recombination, and site-specific recombination (in vivo or in situ).

The vectors of the present invention can be used to express cDNA clones. Accordingly, cDNA (full-length or cDNA fragments) is inserted into the vectors of the invention by ligation or other methods known in the art. In this embodiment, it can be useful to include in the vector a splice acceptor site at the 5′ end of the cDNA molecule. The splice acceptor site, when suitably positioned adjacent to the cDNA copy of the gene of interest, allows the gene to be efficiently expressed to produce the protein of interest. Since cDNA molecules do not normally contain functional splice acceptor sequences, the vector-encoded splice acceptor site allows the upstream exons to be spliced to the cDNA to produce a chimeric mRNA molecule capable of being translated into the protein encoded by the cDNA (FIG. 4).

The vectors of the present invention can be used to express genes encoded by genomic DNA or fragments thereof (FIG. 5). Accordingly, genomic DNA can be inserted into the vectors of the invention by ligation or other methods known in the art. Alternatively, vectors of the invention can be inserted into cloned genomic DNA using in vitro transposition or retroviral insertion. Methods for in vitro transposition and retroviral insertion have been described previously (U.S. patent application Ser. No. 09/276,820, incorporated herein by reference for these methods).

As described for the cDNA expression vectors above, a splice acceptor site can be engineered into the vector adjacent to the genomic fragment. This is particularly important for single exon genes since this class of gene does not contain a functional splice acceptor site. As a result, a splice acceptor site can be engineered into or upstream of a single exon gene, as described for expression of cDNA clones. Generally however, when genes are expressed from genomic DNA, a splice acceptor site does not need to be engineered into the vector. Instead, the promoter/exon units can simply splice to the first downstream splice acceptor site flanking an exon of the gene of interest. This is made possible by the fact that most eukaryotic genes are segmented into exons. Located between each exon is an intron; each intron is flanked by a splice donor site and a splice acceptor site at its 5′ and 3′ end, respectively. By using a vector that does not contain a splice acceptor site between the promoter/exon units and the genomic fragment, splicing will occur directly from each promoter/exon unit to the first genomic DNA encoded splice acceptor site to produce a mRNA molecule. Since each vector encoded exon can be designed to be in frame with the open reading frame of the genomic DNA encoded gene, high levels of protein expression can be achieved.

The vectors of the present invention can also be used to express a DNA sequence that does not correspond to a cDNA or a genomic DNA sequence, i.e., is not naturally occurring. Accordingly, this encompasses chemically synthesized nucleic acid molecules as well as antisense nucleic acid molecules and ribozymes.

The vectors of the invention can also be used to activate endogenous genes in situ by non-homologous recombination. In this embodiment, the vectors are inserted into a host cell genome. The insertion can be at spontaneous chromosome breaks present in the cell. Alternatively, the vectors can be integrated into chromosome breaks induced by treating cells with DNA breaking agents. Useful DNA breaking agents include, but are not limited to, radiation, free radicals, and nucleases. Methods of activating endogenous genes by non-homologous recombination have been described in detail (U.S. patent application Ser. No. 09/276,820, incorporated herein by reference for these methods).

Vectors of the invention can also be inserted into the genome of a host cell by other forms of non-homologous recombination, such as retroviral integration or transposition. Methods for making retroviral vectors are well known in the art, and vectors and packaging cell lines are commercially available, for example, from CloneTech, Palo Alto, Calif. In this embodiment of the invention, the vectors can contain retroviral LTRs and/or packaging sequences. In embodiments of the invention involving transposition, vectors will contain appropriate transposition sequences, as are well-known in the art. Examples of vectors containing retroviral LTRs and packaging signals or transposition signals are shown in FIGS. 8 and 10.

The vectors of the present invention can be used to activate endogenous genes using homologous recombination. To practice this embodiment of the invention, the vectors should contain one or more homologous targeting sequences. As defined herein, a targeting sequence is any polynucleotide sequence capable of directing site-specific integration, by homologous recombination, of the vector into the genome of a host cell. In general, when linear vectors are introduced into a host cell, the vector will contain two targeting sequences that flank the functional elements of the vector. An example of one type of vector containing targeting sequences is shown in FIG. 9. Alternatively, when circular vectors are introduced into a host cell, the vector can contain a single targeting sequence. The configuration of targeting sequences on gene activation vectors and general methods for activating endogenous genes using homologous recombination have been described previously (2-6).

Vectors of the invention can be inserted into or next to a gene or nucleotide sequence using site-specific recombination. Site-specific recombination involves the exchange of genetic material at predetermined sites, designated by specific DNA sequences present on both recombining molecules. In this reaction, a protein recombinase binds to the recombination signal sequences, creates strand scission, and facilitates DNA strand exchange. Thus, in this embodiment, one or more recombination signals can be incorporated onto the vector (FIG. 11). A variety of site-specific recombination systems, along with their recombination signal and recombinases, are known in the art including, but not limited to Cre-lox recombination, V(D)J recombination, Flp recombination, Hin recombination, lambda phage integration. In vitro and in vivo assays and applications using site specific recombination have been described (refs 23-28, all of which are incorporated herein by reference). Based on the description of the present invention, references incorporated herein, and published manuscripts, a person of skill in the art would recognize how to make and use multi-promoter/exon vectors capable of site specific recombination.

The vectors of the invention can encode signal peptides, partial signal peptides, epitope tags, and other useful sequences known in the art.

The vectors of the present invention can also be used to modify the gene or protein of interest. For example, a secretion signal sequence can be included on the expression construct to facilitate the secretion of the gene of interest. In some cases, depending on the intron/exon structure of the gene of interest, the secretion signal sequence can replace all or part of the signal sequence of the endogenous gene. In other cases, the signal sequence will allow a protein that is normally located intracellularly to be secreted. To express and secrete proteins from multi-exon genes in which a portion of their signal peptide is encoded in exon I and a portion in exon II, vectors comprising promoter/exon units encoding partial signal sequences can be used. Upon splicing to exon II, sequences in the activation exon replace missing signal sequences, thereby allowing the protein to be secreted from these genes.

The vectors of the present invention can be used to express a gene as a full length protein, or as a truncated biologically active form of the protein. Expression of truncated genes capable of causing dominant-negative phenotypes in a cell is also possible. The vectors can also be used to express biologically inactive proteins and peptides, useful as antigens, for example.

### BRIEF DESCRIPTION OF THE DRAWINGS

FIG. 1. Schematic diagram of a multi-promoter/exon expression construct. The vector is shown schematically in its circular form. Three promoter/exon units are depicted. The arrows denote promoter sequences. The activation exons are shown as open boxes and splice donor sequences are indicated by S/D. While not required for all embodiments of the invention, a suitable restriction site is shown downstream of the multi-promoter/exon units. This site can be used to linearize the vector or to clone a gene of interest into the vector. An exemplary antibiotic resistance gene, β-lactamase, and plasmid origin or replication, PBR322 ori, are shown.

FIG. 2. Schematic diagram of multi-promoter/exon expression constructs. Polynucleotide sequences are shown schematically in their linear form. A different number of promoter/exon units is shown in each vector. In FIGS. 2A, 2B, and 2C, the vectors contains two, three, and four promoter/exon units, respectively. The arrows denote promoter sequences. The activation exons are shown as open boxes and the splice donor sequences are indicated by S/D.

FIG. 3. Schematic diagram of a multi-promoter/exon expression construct. The vector is shown schematically in its circular form. Three promoter/exon units are depicted. The arrows denote promoter sequences. The activation exons are shown as open boxes and splice donor sequences are indicated by S/D. While not required for all embodiments of the invention, a multi-cloning site, designated MCS, is shown downstream of the multi-promoter/exon units. This site can be used to linearize the vector or to clone a gene of interest into the vector. A splice acceptor site, designated S/A, is shown immediately next to the MCS. Following insertion of a gene into the MCS and introduction into a suitable host cell, the splice acceptor site allows splicing to occur from any upstream promoter/exon unit, thereby removing intervening sequences and allowing the resulting transcript to be translated into a protein of interest.

FIG. 4. Schematic diagram of gene expression from a multi-promoter/exon vector containing a cDNA insert. Polynucleotide sequences are shown schematically in linear form. The vector construct can be operably linked to the gene of interest by cloning/ligation. The arrows on the polynucleotide sequence denote promoter sequences. The activation exons are shown as shaded boxes and the splice donor sequences are indicated by S/D. The gene of interest is shown as an open box downstream of the multi-promoter/exon units. To allow splicing from the promoter/exon units to the cDNA encoded gene, a splice acceptor site, designated by S/A, has been included on the vector immediately upstream of the gene of interest. Following introduction into a suitable host cell, transcription and splicing occur to produce multiple RNA molecules, each generated from a different promoter/exon unit. Subsequent translation will result in the production of the protein of interest from each transcript.

FIG. 5. Schematic diagram of gene expression from a multi-promoter/exon vector. Polynucleotide sequences are shown schematically in linear form. The vector construct is operably linked to the gene of interest by cloning/ligation, cotransfection, non-homologous recombination, site-specific recombination, homologous recombination, or other methods described herein. The arrows on the polynucleotide sequence denote promoter sequences. The activation exons are shown as shaded boxes and the splice donor sequence is indicated by S/D. The gene of interest is shown downstream of the multiple promoter/exon units. In this example, the gene is encoded by multiple exons, designated by open boxes and labeled with roman numerals. Each exon, except exon I, is flanked by a splice acceptor site, designated by S/A. Once the vector has been operably linked to the gene of interest in a suitable host cell, transcription and splicing occur to produce multiple RNA molecules, each generated from a different promoter/exon unit. Subsequent translation will result in the production of the protein of interest from each transcript. While not shown in this example, the vector can also contain an amplifiable marker. This allows gene expression to be further enhanced via gene amplification. Other genetic elements can also be included on the vector as described herein.

FIG. 6. Schematic diagram of a multi-promoter/exon expression construct containing selectable markers. Polynucleotide sequences are shown schematically in linear form. The arrows denote promoter sequences. The activation exons are shown as open boxes and the splice donor sequence is indicated by S/D. The selectable marker is shown operably linked to a promoter sequence. (A) The selectable marker on the vector contains a polyadenylation signal, designated by pA, and is located upstream of the multi-promoter/exon units. In this example, the selectable marker is oriented to drive expression toward the multi-promoter/exon units. While not shown, the selectable marker can alternatively be oriented to transcribe away from the multi-promoter/exon units. (B) Poly (A) Trap: The selectable marker lacks a polyadenylation signal and optionally contains a splice donor site located at its 3′ end. In this example, the selectable marker is located upstream of the multi-promoter/exon units and is oriented to drive expression toward the multi-promoter/exon units. (C) Poly (A) Trap: The selectable marker lacks a polyadenylation signal and optionally lacks a splice donor site at its 3′ end. In this example, the selectable marker is located downstream of the multi-promoter/exon units and is oriented to drive expression toward the multi-promoter/exon units.

FIG. 7. Schematic diagram of a multi-promoter/exon expression constructs containing an amplifiable marker. Polynucleotide sequences are shown schematically in their linear form. The arrows denote promoter sequences. The activation exons are shown as open boxes and the splice donor sequence is indicated by S/D. The amplifiable marker is shown operably linked to a promoter sequence. The amplifiable marker can also contain a poly (A) signal designated pA. Alternatively, the amplifiable marker can lack a poly (A) signal and can contain a splice donor site at its 3′ end (shown in FIG. 6).

FIG. 8. Schematic diagram of a retroviral multi-promoter/exon expression construct. Polynucleotide sequences are shown schematically in their linear form. The arrows denote promoter sequences. The activation exons are shown as open boxes and the splice donor sequence is indicated by S/D. 5′ and 3′ LTRs are shown flanking the vector. A packaging signal is indicated by a Ψ located downstream of the 5′ LTR.

FIG. 9. Schematic diagram of a multi-promoter/exon expression construct useful for homologous recombination/targeting to a gene of interest. Polynucleotide sequences are shown schematically in their linear form. The polynucleotide sequence on the top represents a targeting vector and the polynucleotide sequence at the bottom represents an endogenous gene. Cross-over lines are shown between the targeting sequences on the vector and the endogenous gene locus to illustrate the strand exchange reaction that occurs during homologous recombination. The arrows on the polynucleotide sequences denote promoter sequences. The activation exons are shown as shaded boxes. Splice donor sequences and splice acceptor sequences are indicated by S/D and S/A, respectively. Several exons from the endogenous gene are shown and are labeled with Roman numerals. Targeting sequences are shown flanking the multi-promoter/exon sequences on the vector. Typically, targeting sequences are derived from the locus in and/or around the gene of interest. While not shown, the vectors optionally contain selectable and amplifiable markers. Following homologous recombination in a host cell, the multi-promoter/exon units become operably linked to the endogenous gene, thereby activating its expression.

FIG. 10. Schematic diagram of a transposon multi-promoter/exon expression construct. Polynucleotide sequences are shown schematically in their linear form. The arrows denote promoter sequences. The exonic sequences are shown as open boxes and splice donor sequences are indicated by S/D. Transposon signals are shown as fill triangles flanking the multipromoter exon units. Upon treatment with a transposase and a target nucleic acid, all of the polynucleotide sequences between and including the transposon signals will be integrated into the target nucleic acid. While not shown, the vectors may optionally contain selectable and amplifiable markers, which when present, would be positioned between the transposon signals in any of the configurations shown in FIGS. 6 and 7, or as described herein. Also, as described herein, integration of transposon vectors into a target polynucleotide can also be performed in situ.

FIG. 11. Schematic diagram of a multi-promoter/exon expression construct capable of site specific recombination. The multi-promoter/exon vector is shown at the bottom of the figure in its circular form. A multi-exon gene is shown as a linear molecule to illustrate an endogenous gene. The same multi-exon gene is also shown as a cloned genomic fragment in a vector (illustrated as a hatched circle). While not shown in this example, the gene of interest may also be a cloned cDNA present in a vector containing a site-specific recombination signal. The arrows denote promoter sequences. The activation exons are shown as shaded boxes. Splice donor and splice acceptor sequences are indicated by S/D and S/A, respectively. Lox P recombination signals are shown as a filled triangle flanking the multi-promoter/exon units and gene of interest. Upon treatment with Cre recombinase, all of the polynucleotide sequences on the vector between and including the recombination signals will be integrated into the recombination signal on the nucleic acid containing the gene of interest. While not shown, the vectors may optionally contain selectable and amplifiable markers. As described herein, integration of site specific recombination vectors into a target polynucleotide can be performed in vitro or in situ.

FIG. 12. Schematic diagram of pRIG-MP1. The vector is shown schematically in its circular form. Three promoter/exon units are depicted. The arrows denote promoter sequences. The type of each promoter is indicated. The activation exons are shown as open boxes and splice donor sequences are indicated by S/D. In this vector, the activation exons do not encode a translation start codon. While not required for all embodiments of the invention, a unique restriction site, Bam HI, is shown downstream of the multi-promoter/exon units. This site may be used to linearize the vector or to clone a gene of interest into the vector. A selectable marker, neomycin resistance gene, and an amplifiable marker, dihydrofolate reductase, are also shown. Both markers are expressed from a promoter element, as indicated, and followed by a polyadenylation signal (not shown). A pUC plasmid origin of replication and an antibiotic resistance gene, β lactamase, are also present.

FIG. 13. Nucleotide sequence for pRIG-MP1.

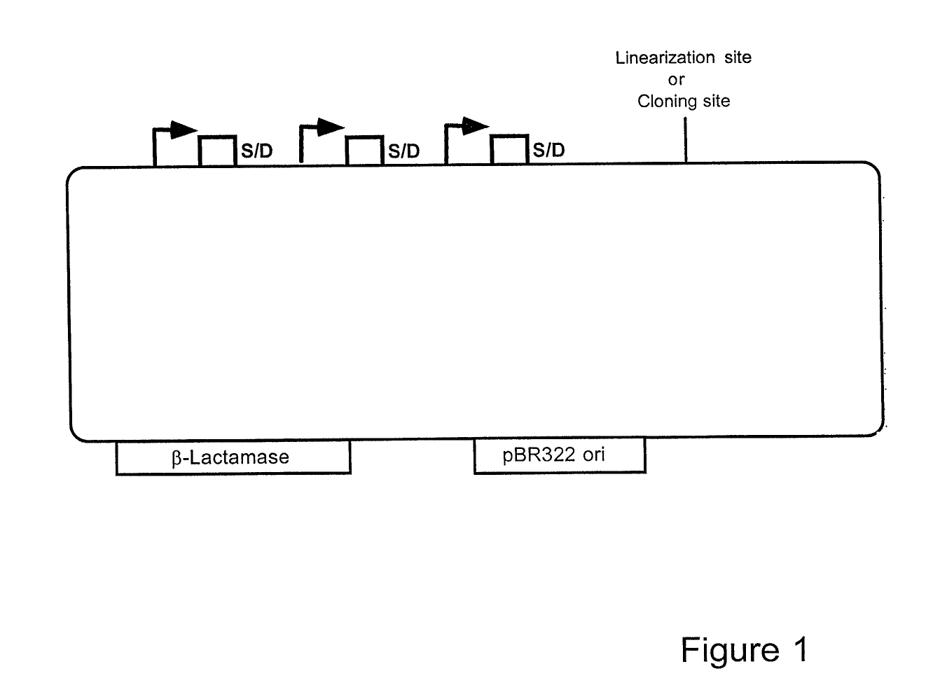

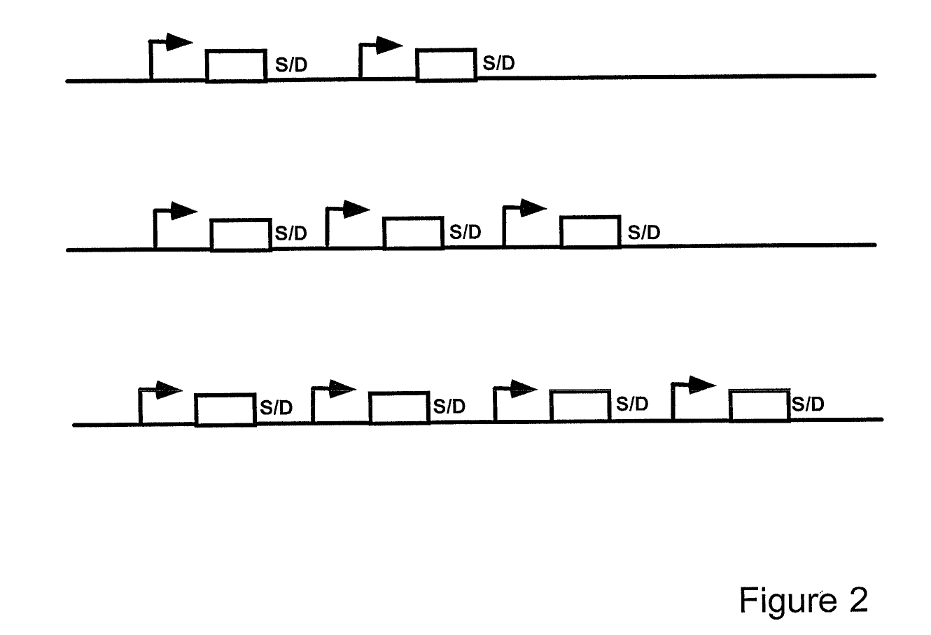

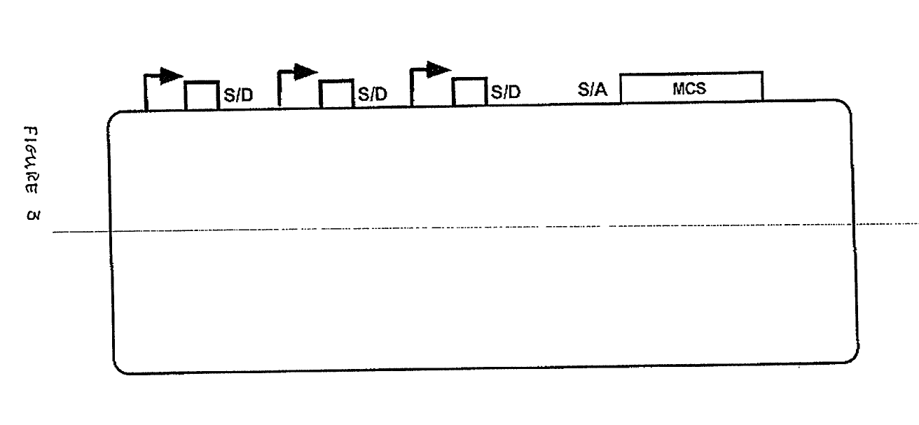

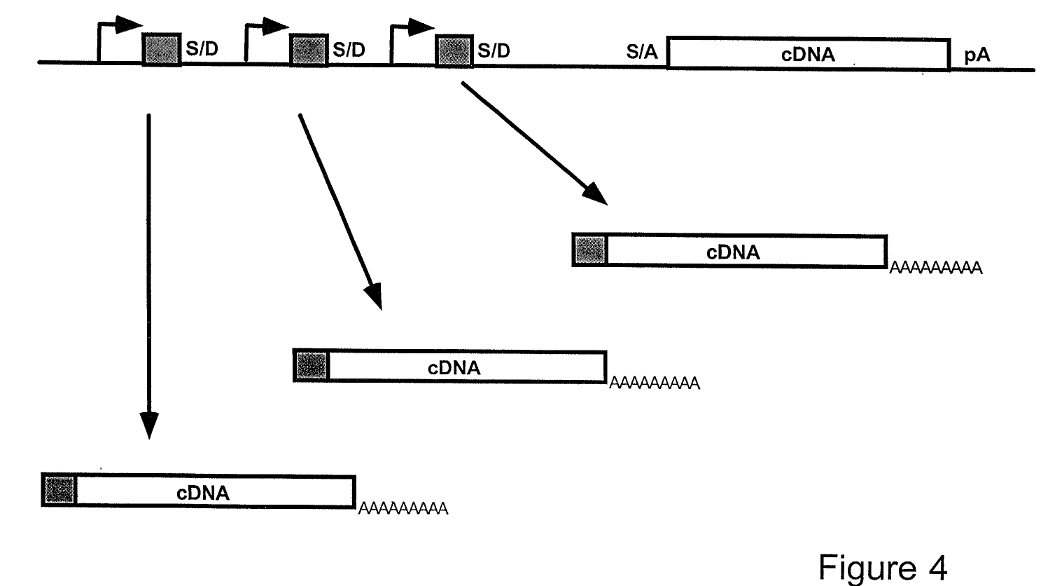

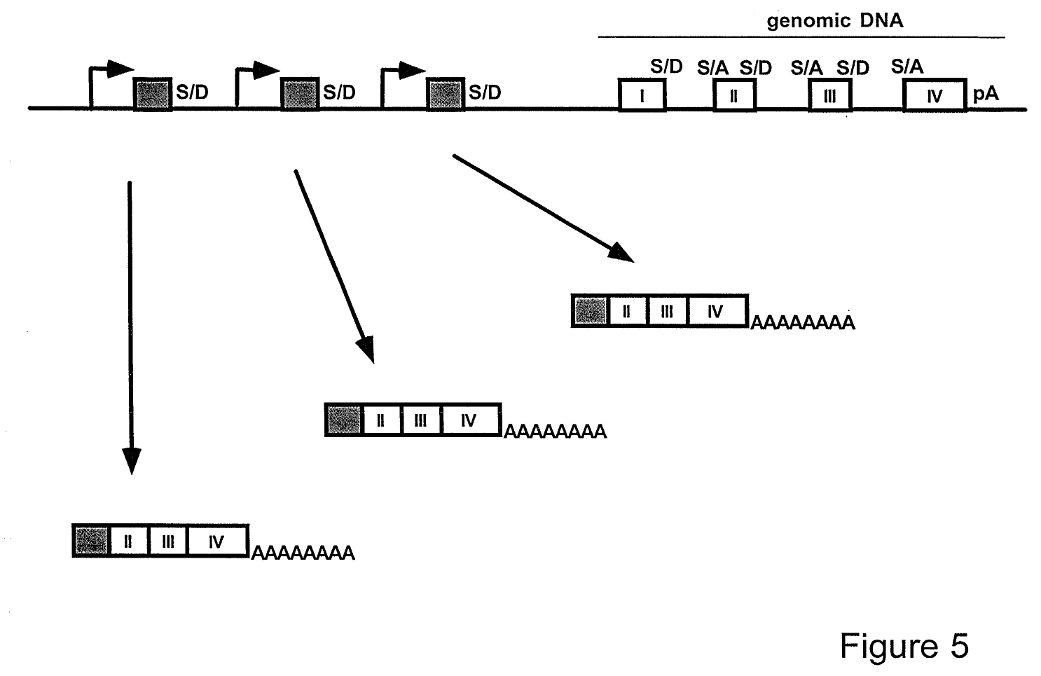

While not necessarily shown in the figures, the vectors can also contain selectable markers, amplifiable markers, and other genetic elements as described herein.

### DETAILED DESCRIPTION OF THE INVENTION

The Multi-Promoter/Exon Expression Vector The vectors of the invention can be generally characterized as having multiple transcriptional regulatory sequences, each operably linked to an exon and splice donor site. The transcriptional regulatory sequence can comprise a variety of genetic elements, but includes, at least, a promoter element. Thus, the operable linkage between the regulatory sequence and exon is designated herein as a promoter/exon unit.

The promoter/exon units are arranged on the vector in such a manner so as to allow a gene of interest to be expressed from multiple promoter/exon units. All units direct transcription of the same strand, i.e., face in the same direction. In one embodiment, the promoter/exon units are arranged in tandem and a gene of interest is operably positioned downstream of and in the same orientation as the promoter/exon units. That is, one or more promoter/exon units are arranged adjacent to each other. In another embodiment, one or more promoter/exon units is separated by a spacer nucleotide sequence.

The gene of interest can be operably linked to the promoter/exon units by any method, such as standard nucleic acid ligation. Furthermore, the operable linkage can be created in vitro or in situ (within a cell). Examples of methods useful for creating an operable linkage between a gene or cDNA and the vector are discussed herein, but are not meant to be limiting in any way.

The vectors of the invention can contain any number of such multiple transcriptional regulatory sequences, for example, 1-5, 5-10, 10-15, 15-20, or more of these sequences. Moreover, the promoters or other transcriptional regulatory sequences can be identical or different. For example, promoters can be derived from the same or different genes. As an example, not meant to be limiting, all promoters could be derived from CMV immediate early promoter or one or more CMV immediate early gene promoters is combined with one or more promoters derived from another source.

The characteristics of the regulatory sequences, exon, and splice donor sites are described below. Furthermore, while the vectors of the invention can solely comprise two or more promoter/exon units, additional genetic elements can also be present on the vector, as described below.

Transcriptional Regulatory Sequences

The vectors of the invention contain a transcriptional regulatory sequence operably linked to each activation exon and splice donor site. The regulatory sequence can contain a variety of genetic elements, as described below. However, at a minimum, the regulatory sequence contains a promoter.

As used herein, a promoter is any polynucleotide sequence, alone or in combination with other polynucleotide sequences, capable of initiating transcription.

The promoter can be derived from a naturally-occurring polynucleotide sequence. Alternatively, the promoter can be an engineered polynucleotide sequence. Examples of engineered promoters have been described in the literature. In addition, the promoter can be a polynucleotide sequence that normally is not capable of initiating transcription, however, when used in combination with engineered transcription factors, becomes capable of initiating transcription. Thus, the transcriptional regulatory sequence can comprise one or more artificial transcription factor binding sites. Examples of modified and/or artificial transcription factors have been described previously (13, 14).

The promoter can be a constitutive promoter. Alternatively, the promoter can be inducible. Use of inducible promoters will allow low basal levels of activated protein to be produced by the cell during routine culturing and expansion. The cells can then be induced to produce large amounts of the desired proteins, for example, during manufacturing or screening. Examples of inducible promoters include, but are not limited to, the tetracycline-inducible promoter and the metallothionein promoter.

The regulatory sequence, for example the promoter or an enhancer, on the vector can be isolated from cellular or viral genomes. Examples of cellular regulatory sequences include, but are not limited to, regulatory elements from the actin gene, metallothionein I gene, immunoglobulin genes, casein I gene, serum albumin gene, collagen gene, globin genes, laminin gene, spectrin gene, ankyrin gene, sodium/potassium ATPase gene, and tubulin gene. Examples of viral regulatory sequences include, but are not limited to, regulatory elements from Cytomegalovirus (CMV) immediate early gene, adenovirus late genes, SV40 genes, retroviral LTRs, and Herpesvirus genes. Typically, regulatory sequences contain binding sites for transcription factors such as NF-kB, SP-1, TATA binding protein, AP-1, and CAAT binding protein.

In a preferred embodiment, the promoter is a viral promoter. In a particularly preferred embodiment, the promoter is the CMV immediate early gene promoter. In alternative embodiments, the promoter is a cellular, non-viral promoter.

In preferred embodiments, the regulatory element contains an enhancer. In particularly preferred embodiments, the enhancer is the cytomegalovirus immediate early gene enhancer. In alternative embodiments, the enhancer is a cellular, non-viral enhancer.

The transcriptional regulatory sequence can also comprise one or more scaffold-attachment regions or matrix attachment sites, negative regulatory elements, and transcription factor binding sites. Regulatory sequences can also include locus control regions.

Activation Exon and Splice Donor Site

Transcriptional regulatory sequences are positioned upstream of each activation exon sequence and splice donor site to produce a promoter/exon unit. As used herein, the activation exon comprises any nucleotide sequence between the transcription start site and the first downstream splice donor site, including the first three nucleotides of the splice donor site.

The terms upstream and downstream, as used herein, are intended to mean in the 5′ or in the 3′ direction, respectively, relative to the transcribed strand.

A splice donor site is a polynucleotide sequence capable, in combination with a splice acceptor site, of directing the removal of an intron from a RNA transcript. The consensus sequence for splice donor sites is (A/C)AG GURAGU (where R represents a purine nucleotide) with nucleotides in positions 1-3 located in the exon and nucleotides GURAGU located in the intron. Additional splice donor sequences have been described and these, along with other splice donor sequences known to those of skill in the art or obtained through routine experimentation, can be used in the vectors of the present invention. Since the splice donor sequence requires a splice acceptor site for splicing to occur, a splice acceptor site must be in sufficient proximity to the gene of interest such that the gene of interest is translated. As discussed below, the gene sequence can contain a splice acceptor site. Alternatively, a splice acceptor site can be engineered into the vector or gene of interest.

The vectors of the present invention are generally characterized as containing more than one promoter/exon unit capable of expressing an operably linked polynucleotide sequence in its functional form. As used herein, the term “expressed in its functional form” means that the polynucleotide is transcribed to produce RNA, which in turn, can be spliced to produce a mature RNA molecule capable of carrying out its intended function. For example, where a protein or peptide is the desired expression product, the vector will be capable of expressing mature RNA molecules that can then be translated into the protein of interest. Alternatively, where expression of a ribozyme is the desired expression product, the vector will be capable of expressing a ribozyme in its enzymatically active form. and transcription factor binding sites. Regulatory sequences can also include locus control regions.

Activation Exon and Splice Donor Site

Transcriptional regulatory sequences are positioned upstream of each activation exon sequence and splice donor site to produce a promoter/exon unit. As used herein, the activation exon comprises any nucleotide sequence between the transcription start site and the first downstream splice donor site, including the first three nucleotides of the splice donor site.

The terms upstream and downstream, as used herein, are intended to mean in the 5′ or in the 3′ direction, respectively, relative to the transcribed strand.

A splice donor site is a polynucleotide sequence capable, in combination with a splice acceptor site, of directing the removal of an intron from a RNA transcript. The consensus sequence for splice donor sites is (A/C)AG GURAGU (where R represents a purine nucleotide) with nucleotides in positions 1-3 located in the exon and nucleotides GURAGU located in the intron. Additional splice donor sequences have been described and these, along with other splice donor sequences known to those of skill in the art or obtained through routine experimentation, can be used in the vectors of the present invention. Since the splice donor sequence requires a splice acceptor site for splicing to occur, a splice acceptor site must be in sufficient proximity to the gene of interest such that the gene of interest is translated. As discussed below, the gene sequence can contain a splice acceptor site. Alternatively, a splice acceptor site can be engineered into the vector or gene of interest.

The vectors of the present invention are generally characterized as containing more than one promoter/exon unit capable of expressing an operably linked polynucleotide sequence in its functional form. As used herein, the term “expressed in its functional form” means that the polynucleotide is transcribed to produce RNA, which in turn, can be spliced to produce a mature RNA molecule capable of carrying out its intended function. For example, where a protein or peptide is the desired expression product, the vector will be capable of expressing mature RNA molecules that can then be translated into the protein of interest. Alternatively, where expression of a ribozyme is the desired expression product, the vector will be capable of expressing a ribozyme in its enzymatically active form.

To express a polynucleotide sequence, the promoter/exon units are operably linked to the polynucleotide sequence. Operably linked is defined as a configuration that allows transcription through the designated sequence(s). An operable linkage between the promoter/exon units and a polynucleotide sequence can be created by cloning/ligation, homologous recombination, nonhomologous recombination, site specific recombination, or other methods disclosed herein or known in the art.

Once an operable linkage is created between the promoter/exon units and a polynucleotide sequence of interest, if not already in a host cell (i.e., as when ligation of exogenously-introduced sequences and vector occurs), the vector is introduced into a suitable host cell. In the cell, each promoter facilitates transcription initiation, at a site generally referred to as a CAP site. Transcription then proceeds through the adjacent activation exon and polynucleotide of interest to produce a primary transcript. Splicing of primary transcripts, the process by which introns are removed, occurs between the splice donor site adjacent to the upstream-most activation exon and the first downstream splice acceptor site. If the promoter initiating transcription is an upstream promoter in the vector, then downstream promoter/exon units can be part of the primary transcript; however, these downstream promoter/exon units are removed during RNA splicing. The result of splicing is the creation of a mRNA molecule with a single activation exon at its 5′ end, followed immediately by the polynucleotide of interest.

The activation exon can lack a translation start codon. Vectors containing activation exons lacking a translation start codon are useful for expressing protein from genes that contain a translation start codon. Following transcription, the activation exon (lacking a start codon) would be spliced to the gene of interest (containing a translation start codon). This allows cellular translation machinery to initiate protein synthesis from a start codon in the operably linked gene. Thus, in this configuration, the activation exon encodes all or part of the 5′ untranslated region of the RNA.

Alternatively, a translation start codon can be present on the activation exons in each promoter/exon unit.). The translation start codon is usually ATG and preferably an efficient translation initiation site (Kozak, J. (1987) Mol Biol. 196:947). Vectors containing activation exons that encode a translation start codon are useful for expressing protein from genes that lack a translation start codon. When present, the translation start codon on the activation exon is preferably positioned in the same reading frame (relative to the splice donor site) in all exons on the vector, and in the same reading frame as the gene of interest (following splicing). This allows cellular translation machinery to initiate protein synthesis from the start codon in the activation exon and to proceed through the open reading frame of the operably linked gene. When the reading frame of the gene of interest is not known, several different multi-promoter/exon vectors can be tested, each vector comprising activation exons encoding a start codon in a different reading frame. In addition, a vector comprising activation exons lacking a translation start codon would also be useful for protein expression from genes with unknown sequence or structure. Use of vectors, each capable of expressing protein from a gene with a different structure or reading frame, is useful for the creation of, for example, protein expression libraries through cDNA or genomic DNA cloning, or non-homologous recombination in situ.

In vectors containing a translation start codon in the activation exon, additional codons can be located between the translational start codon and the splice donor site. For example, the activation exon can encode a signal secretion signal, a partial signal secretion signal, epitope tag, transmembrane domain, protein domain, selectable marker, screenable marker, or amino acid sequences that reconstitute missing amino acid sequence from the gene of interest. When present, a signal secretion sequence allows the protein of interest to be secreted from the cell. The signal sequence can be used to direct secretion of a protein that is normally secreted. Alternatively, the signal sequence can be used to direct secretion of a protein that is normally intracellular. When present, a partial signal secretion sequence can be to complement a partial signal sequence present in the gene of interest. Accordingly, the partial signal sequence can be any amino acid sequence capable of complementing a partial signal sequence from a gene of interest to produce a functional signal sequence. This vector is particularly useful for replacing signal sequences present in exon I of many genes, since exon I does not contain a splice acceptor site at its 5′ end, and therefore, can not be joined to an activation exon. To recreate signal peptide activity, the partial signal sequence on the activation exon can encode between one and one hundred amino acids, and can be derived from existing genes, or can consist of novel sequences.

The activation exon can be a naturally occurring sequence or can be non-naturally occurring (e.g., produced synthetically). The exon can contain additional codons following the start codon. These codons can be derived from a naturally occurring gene or can be non-naturally occurring (e.g., i.e. sequences not found in nature). The codons can replace missing codons normally present in the gene of interest. Alternatively, the codons can encode amino acid sequence not normally found in the gene of interest. When the sequences encode an epitope tag, any epitope can be used. Typically, an epitope can be as small as a post translational protein modification (e.g. phospho-tyrosine). More typically, the epitope is encoded by 5 to 10 amino acids, however, larger epitopes including entire proteins can be used. As stated above, other amino acid sequences can be placed on the activation exon to enable specific applications of the invention. Based on the teachings disclosed herein and published elsewhere, a person of skill in the art would recognize additional amino acid sequences useful in the present invention.

The term “gene” is generally intended to refer to a nucleic acid sequence that is capable of producing a protein, i.e., that wholly or partly encodes an amino acid sequence. The sequence can be naturally-occurring, as found in the genome (i.e., genomic DNA) or expressed in the cell in its native form (i.e., mRNA), formed by recombinant methods (i.e., engineered, such as cDNA), or chemically synthesized. However, for the purposes of this invention, the use of this term applies as well to a nucleic acid sequence that does not produce a protein, for example, a sequence that produces a useful complementary nucleic acid (e.g., antisense/ribozyme). Therefore, vectors, methods, uses and the like that are directed to a “gene” can also apply to such a nucleic acid sequence (i.e., unless they specifically apply to coding sequences). The polynucleotide sequence can encode as few as two or more amino acids and as many as 10,000 or more amino acids.

Splice Acceptor Sites

The vectors can contain a splice acceptor site downstream of the promoter/exon units. The consensus sequence for splice acceptor sites is YYYYYYYYYYNYAG (where Y denotes any pyrimidine and N denotes any nucleotide (Jackson, I. J. (1991) Nucleic Acids Research 19:3715-3798). Other functional splice acceptor sites may also be used.

When present, the splice acceptor site is positioned to direct RNA splicing from any one of the upstream splice donor sites to the splice acceptor site, thereby removing all intervening sequences. In general, the splice acceptor site is located immediately adjacent to the gene of interest. If no translation start codon is present in the activation exons, then the splice acceptor site is preferably placed in the 5′ untranslated region of the gene of interest. In this situation, the splice acceptor site is placed close enough to the bona fide translation start codon so that cryptic ATGs are not incorporated into the spliced message. Alternatively, the gene of interest can lack its own translation start codon. In this case, translation start codons can be included in each activation exon, and the splice acceptor site is placed immediately next to the open reading frame of the gene of interest. The splice acceptor site can also be placed upstream of or within the gene of interest. This results in amino acid sequence additions or deletions, respectively.

Selectable Markers

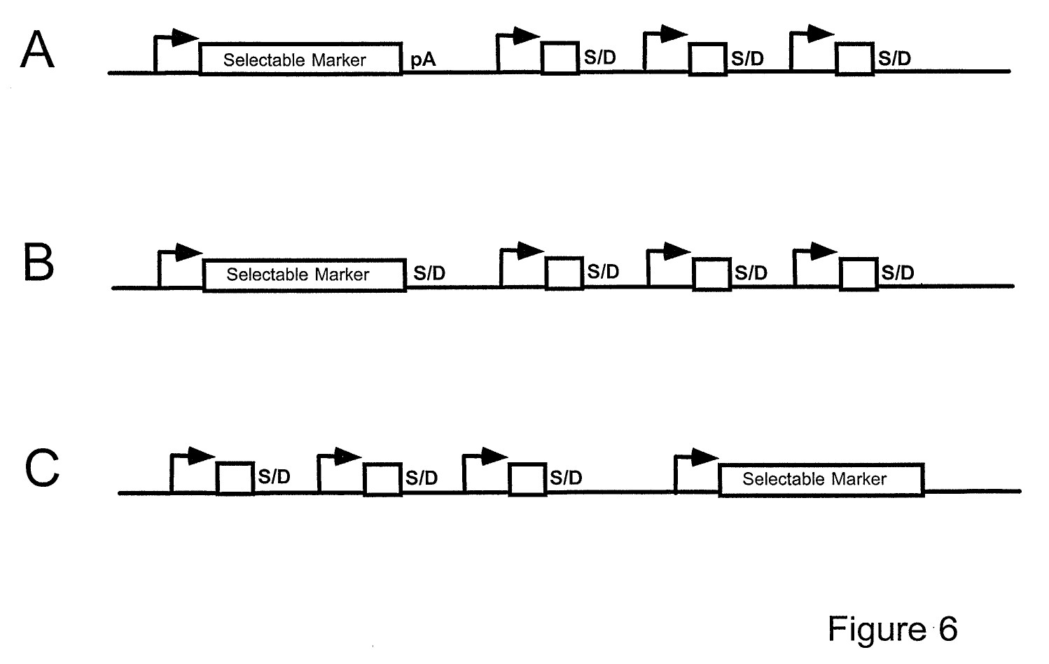

The vector construct can contain a selectable marker to facilitate the identification and isolation of cells containing the vector construct (FIG. 6). Examples of selectable markers include genes encoding neomycin resistance (neo), hypoxanthine phosphoribosyl transferase (HPRT), puromycin (pac), dihydro-orotase glutamine synthetase (GS), histidine D (his D), carbamyl phosphate synthase (CAD), dihyrofolate reductase (DHFR), multidrug resistance 1 (mdr1), aspartate transcarbamylase, xanthine-guanine phosphoribosyl transferase (gpt), and adenosine deaminase (ada).

Alternatively, the vector can contain a screenable marker, in place of or in addition to, the selectable marker. A screenable marker allows the cells containing the vector to be isolated without placing them under drug or other selective pressures. Examples of screenable markers include genes encoding cell surface proteins, fluorescent proteins, and enzymes. The vector containing cells can be isolated, for example, by FACS using fluorescently-tagged antibodies to the cell surface protein or substrates that can be converted to fluorescent products by a vector encoded enzyme.

Alternatively, selection can be effected by phenotypic selection for a trait resulting from expression of the protein of interest. The vector construct, therefore, can lack a selectable marker other than the “marker” provided by the expressed gene. In this embodiment, cells can be selected based on a phenotype conferred by the gene of interest. Examples of selectable phenotypes include cellular proliferation, growth factor independent growth, colony formation, cellular differentiation (e.g., differentiation into a neuronal cell, muscle cell, epithelial cell, etc.), anchorage independent growth, activation of cellular factors (e.g., kinases, transcription factors, nucleases, etc.), expression of cell surface receptors/proteins, gain or loss of cell-cell adhesion, migration, and cellular activation (e.g., resting versus activated T cells).

The selectable marker can contain a poly adenylation (poly (A)) signal.

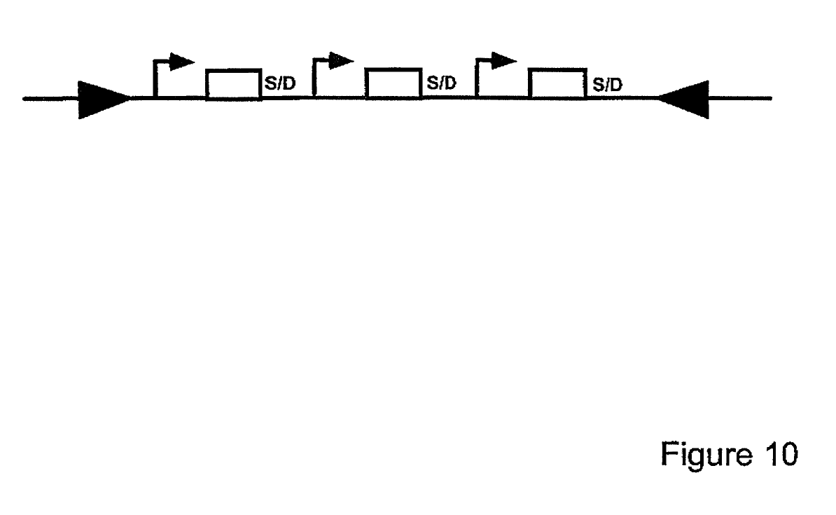

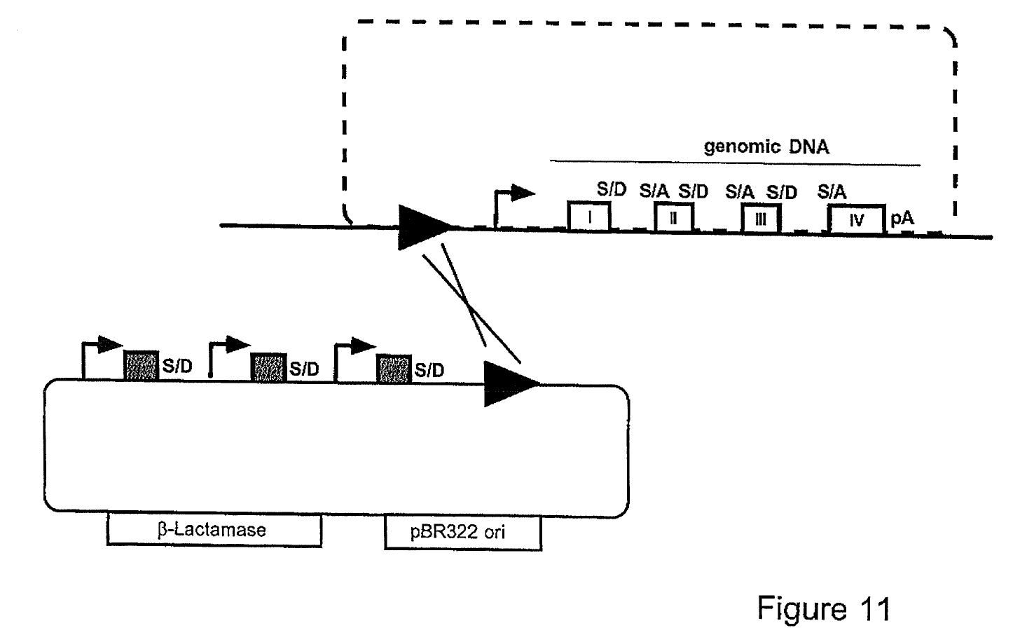

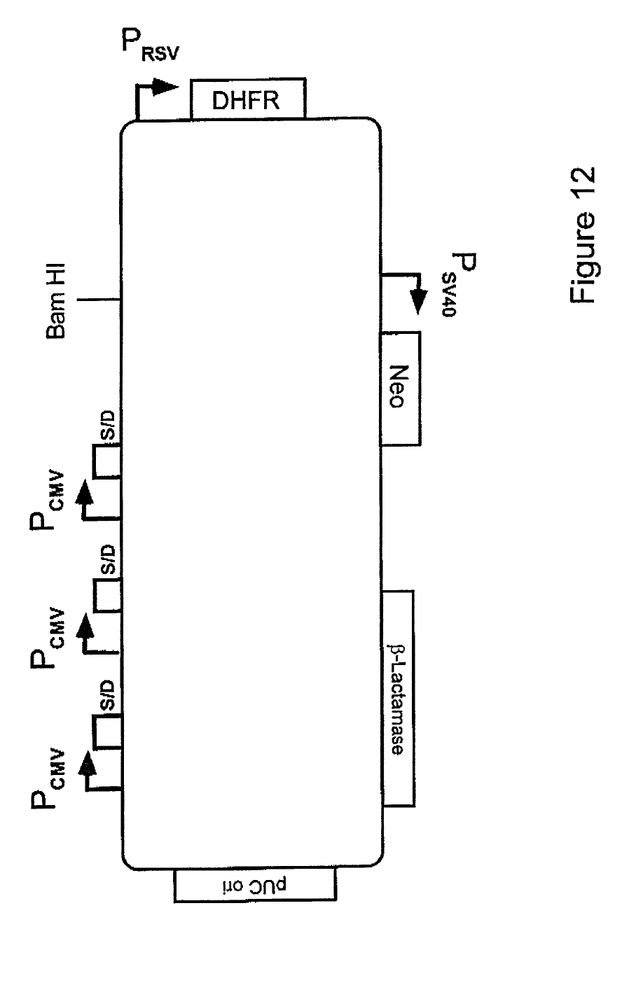

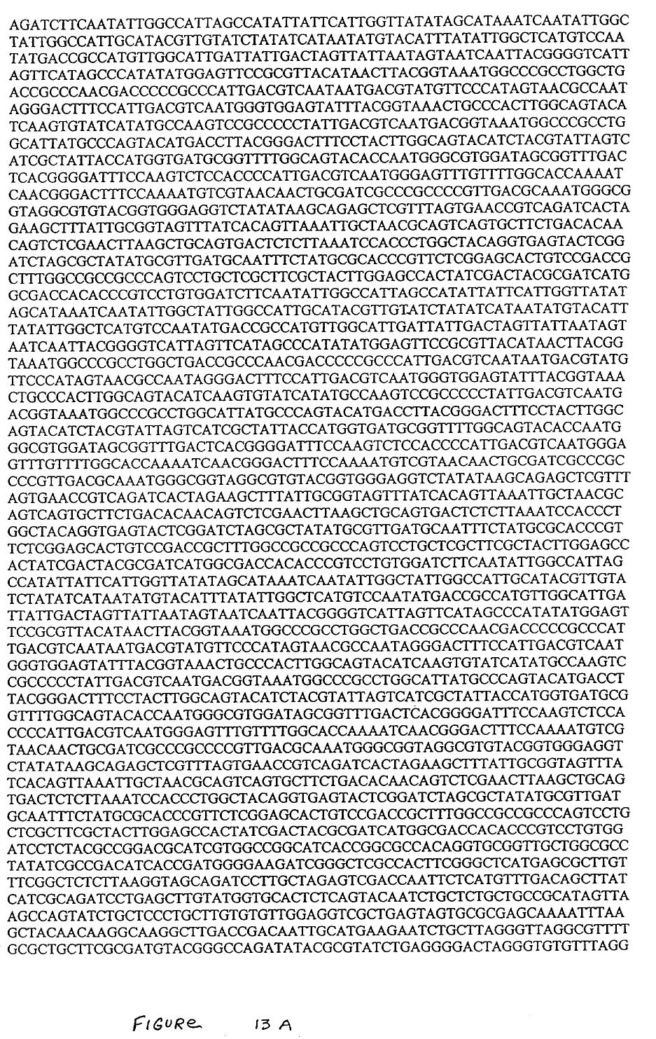

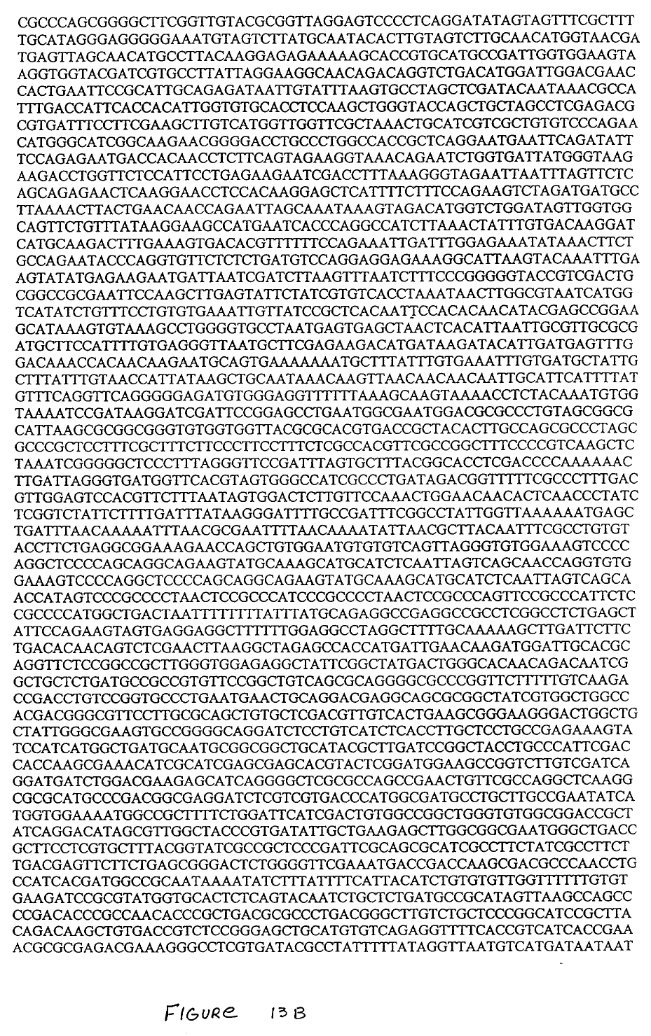

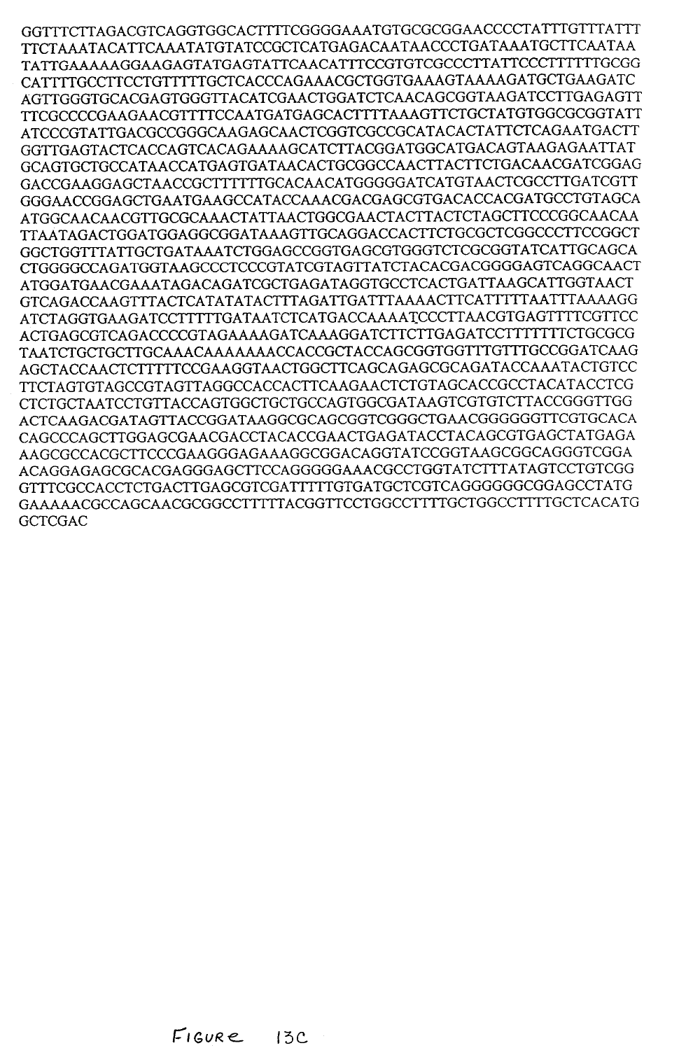

Alternatively, the selectable marker can be configured on the vector to act as a poly (A) trap (FIG. 10). In this embodiment, the selectable marker lacks a poly adenylation signal. To produce a stable mRNA encoding the selectable marker, a poly (A) signal must be acquired from a gene operably linked to the vector. In some applications of the invention (e.g. activation of endogenous genes by homologous or nonhomologous recombination, or expressing genes from isolated genomic fragments), the poly (A) trap vector can be used to select for cells expressing a gene from the vector, and to select against cells that do not express a gene from the vector. The utility of poly (A) trap vectors, along with methods for making and using them, is discussed extensively in U.S. patent application Ser. No. 09/276,820, pages 76-78, incorporated herein by reference for these vectors and methods.

The vectors can contain a negative selectable marker alone, or in combination with a positive selectable marker. Examples of negative selectable markers include hypoxanthine phosphoribosyl transferase (HPRT), thymidine kinase (TK), and diptheria toxin. The negative selectable marker can also be a screenable marker, such as a cell surface protein or an enzyme. Cells expressing the negative screenable marker can be removed by, for example, Fluorescence Activated Cell Sorting (FACS) or magnetic bead cell sorting.

A negative selectable marker can be used to select against undesirable events. For example, a negative selectable marker can be used to select against nonhomologous integration of a vector in applications related to gene activation by homologous recombination (3, 4, 15). A negative selectable marker can also be used with nonhomologous recombination vectors to identify insertion events that lead to activation of multi-exon genes. Other applications for negative selectable markers exist. Vectors containing selectable markers are described herein, and in U.S. patent application Ser. No. 09/276,820 pgs. 78-81, incorporated herein by reference for such vectors.

The selectable marker(s) on the vector can also be configured to create a splice acceptor trap. Like poly (A) trap vectors, a splice acceptor trap may be used in a variety of applications (e.g. activation of endogenous genes by homologous or nonhomologous recombination, or expressing genes from isolated genomic fragments) to select for cells expressing a gene from the vector, and to select against cells that do not express a gene from the vector. The utility of splice acceptor trap vectors, along with methods for making and using these vectors, is discussed extensively in U.S. patent application Ser. No. 09/276,820 pgs. 78-81, incorporated herein by reference for these aspects.

The selectable markers on the vector can be configured to create a dual poly (A)/splice acceptor trap vector. These vectors have a higher degree of specificity when used to select for cells containing the multi-promoter/exon vector in operable linkage with a gene of interest. The utility of splice acceptor trap vectors, along with methods for making and using these vectors, is discussed extensively in U.S. patent application Ser. No. 09/276,820 pg. 81, incorporated herein by reference for these aspects.

To isolate cells that express a positive selectable marker, the cells containing the vector can be placed under the appropriate drug selection. When a positive and negative selectable marker has been included on the vector, selection for the positive selectable marker and against the negative selectable marker can occur simultaneously. In another embodiment, selection can occur sequentially. When selection occurs sequentially, selection for the positive selectable marker can occur first, followed by selection against the negative selectable marker. Alternatively, selection against the negative selectable marker can occur first, followed by selection for the positive selectable marker.

The positive and negative markers are expressed by a transcriptional regulatory element located upstream of the translation start site of each gene. When a positive/negative marker fusion gene or an ires sequence is positioned between the two markers, a single transcriptional regulatory element can drive expression of both markers. A poly(A) signal can be placed 3′ of each selectable marker. If a positive/negative fusion gene is used a single poly(A) signal is positioned 3′ of the markers. Alternatively, a poly(A) signal can be excluded from the vector to provide additional specificity for a gene activation event (see dual poly(A)/splice acceptor trap below).

When present, the selectable marker(s) can be located upstream of the multipromoter/exon units. The selectable marker(s) can be present on the vector in any orientation (i.e. the open reading frame can be present on either DNA strand). When poly (A) trap, splice acceptor trap, or dual poly (A)/splice acceptor trap vectors are used, the selectable marker is positioned to be transcribed on the same strand as the promoter/exon units, and can be positioned relative to the promoter/exon units as described previously (U.S. patent application Ser. No. 09/276,820).

Amplifiable Markers

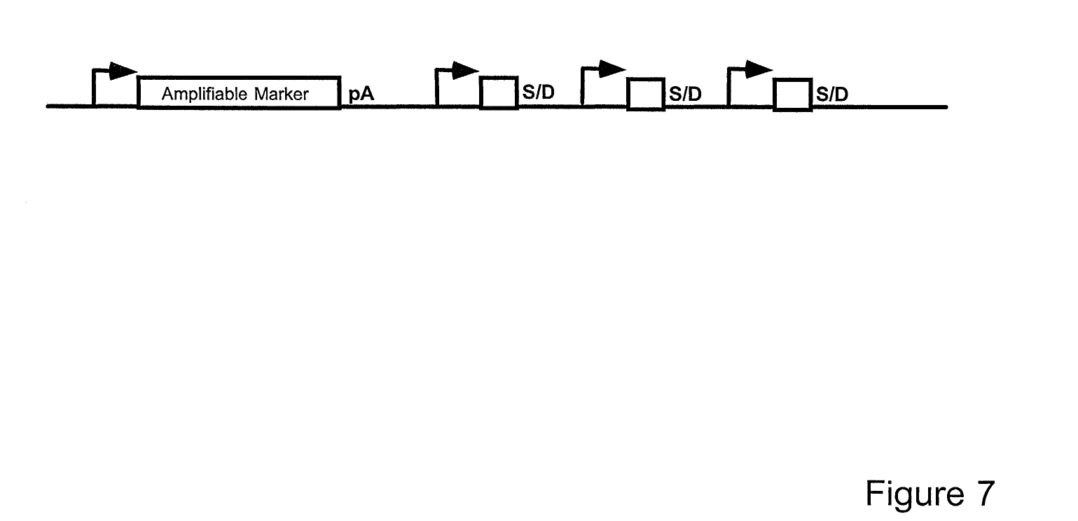

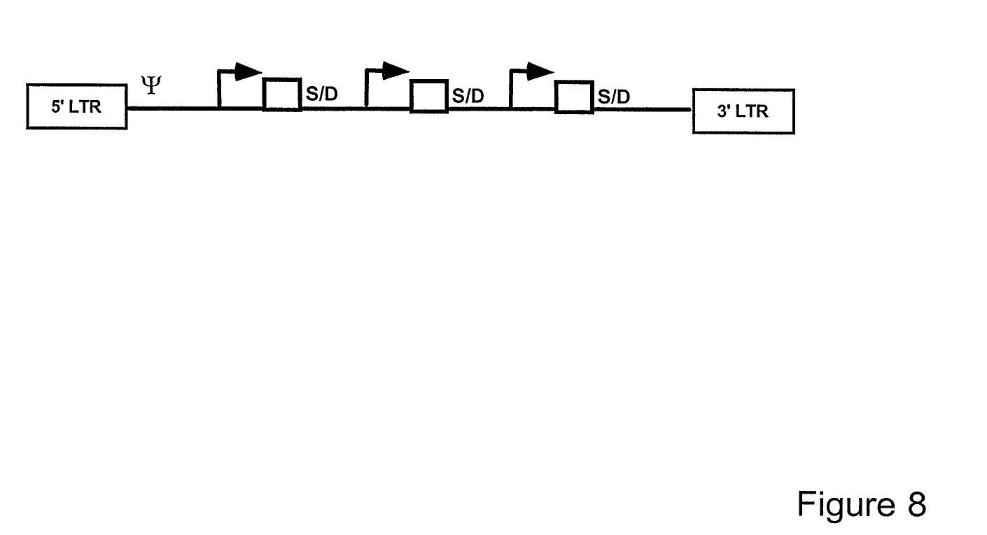

Any vector described herein can include an amplifiable marker (FIG. 7). This enables the vector and the gene of interest to be amplified in copy number, thereby further enhancing expression of the gene of interest. Accordingly, methods of the invention can include a step in which the expressed gene is amplified.

Amplifiable markers are genes that can be selected for higher copy number. Examples of amplifiable markers include dihydrofolate reductase, adenosine deaminase (ada), dihydro-orotase, glutamine synthase (GS), and carbamyl phosphate synthase (CAD). For these examples, the elevated copy number of the amplifiable marker and flanking sequences (including the gene of interest) can be selected for using a drug or toxic metabolite which is acted upon by the amplifiable marker. In general, as the drug or toxic metabolite concentration increases, cells containing fewer copies of the amplifiable marker die, whereas cells containing increased copies of the marker survive and form colonies. These colonies can be isolated, expanded, and analyzed for increased levels of production of the gene of interest.

The presence of an amplifiable marker on the expression construct allows amplification of the marker and any gene in operable linkage to the multipromoter/exon unit. Selection for cells containing increased copy number of the amplifiable marker and gene of interest can be achieved by growing the cells in the presence of increasing amounts of selective agent (usually a drug or metabolite). For example, amplification of dihydrofolate reductase (DHFR) can be selected using methotrexate.

As drug-resistant colonies arise at each increasing drug concentration, individual colonies can be selected and characterized for copy number of the amplifiable marker and gene of interest, and analyzed for expression of the gene of interest. Individual colonies with the highest levels of activated gene expression can be selected for further amplification in higher drug concentrations. At the highest drug concentrations, the clones will express greatly increased amounts of the protein of interest.

When amplifying DHFR, it is convenient to plate approximately 1×10⁷ cells at several different concentrations of methotrexate. Useful initial concentrations of methotrexate range from approximately 5 nM to 100 nM. However, the optimal concentration of methotrexate must be determined empirically for each cell line and integration site. Following growth in methotrexate containing media, colonies from the highest concentration of methotrexate are picked and analyzed for increased expression of the gene of interest. The clone(s) with the highest concentration of methotrexate are then grown in higher concentrations of methotrexate to select for further amplification of DHFR and the gene of interest. Methotrexate concentrations in the micromolar and millimolar range can be used for clones containing the highest degree of gene amplification.

In some embodiments and for certain applications, it may be desirable to place multiple amplifiable markers on the vector. Use of more than one amplifiable marker enables dual selection, or alternatively sequential selection, for each amplifiable marker. This facilitates the isolation of cells that have amplified the vector and gene of interest. Thus, the vector can contain multiple (i.e., two, three, four, five, or more, and most preferably one or two) amplifiable markers to allow for selection of cells containing increased copies of the integrated vector and the adjacent activated endogenous gene.

When present, the amplifiable marker(s) can be located upstream of the multipromoter/exon units. The amplifiable marker(s) can be present on the vector in any orientation (i.e. the open reading frame may be present on either DNA strand).

It is also understood that the amplifiable marker(s) can also be the same gene as the positive selectable marker. Examples of genes that can be used both as positive selectable markers and amplifiable markers include dihydrofolate reductase, adenosine deaminase (ada), dihydro-orotase, glutamine synthase (GS), and carbamyl phosphate synthase (CAD).

Origins of Replication

The vector can contain eukaryotic viral origins of replication useful for gene amplification. These origins can be present in place of, or in conjunction with, an amplifiable marker. Examples of viral origins of replication useful in the present invention include, but are not limited to, SV40 ori, and Epstein Barr ori (Ori P).

Viral origins of replication can also be included on the vector to allow the vector to be maintained in the cell as an episome. Vectors expressing cDNA clones or genomic fragments can be propagated as episomes to allow higher levels of expression.

When viral origins of replication are used, the vectors can be introduced into cells expressing one or more viral replication proteins. Alternatively, viral replication protein(s) can be introduced after the vector is in the cell. Examples of viral replication proteins include, but are not limited to, SV40 T antigen and Epstein Barr virus EBNA-1.

Bacterial Genetic Elements

The vector can also contain genetic elements useful for the propagation of the construct in micro-organisms. Examples of useful genetic elements include microbial origins of replication and antibiotic resistance markers.

Genes, Polynucleotides, Antisense RNA, and Ribozymes

The vector can lack a gene of interest, that is, it does not contain a nucleic acid sequence operably linked to the promoter/exon unit and designed to be transcribed and in some cases translated from that nucleic acid sequence. Such vectors can consist essentially of the multiple promoter/exon units. In this embodiment, the splice donor sites on the vector are said to be unpaired. An unpaired splice donor site is defined herein as a splice donor site present on the expression construct without a downstream splice acceptor site. Thus, vectors lacking a gene of interest are useful, for example, to activate genes by recombination in situ or to serve as vectors capable of accepting and subsequently expressing polynucleotide sequences containing a splice acceptor site.

However, the vector, containing multiple promoter/exon units, can contain a gene of interest. When the vector is operably linked to a gene or polynucleotide sequence containing a splice acceptor site (e.g. a multi-exon gene encoded by genomic DNA), the unpaired splice donor sites become paired with the splice acceptor site. The splice donor site from the vector, in conjunction with the splice acceptor site from the gene, will then direct the excision of all of the sequences between the vector splice donor site and the upstream-most splice acceptor site from the gene of interest. Excision of these intervening sequences removes sequences that interfere with, for example, translation of the protein of interest.

The gene of interest can be a cDNA molecule or a genomic fragment. The gene of interest can also be synthetic (e.g. produced through chemical synthesis). The gene can contain introns and splice acceptor sites. Alternatively, the gene can lack introns and splice acceptor sites.

The gene of interest can encode a poly (A) signal. Alternatively, a heterologous poly (A) signal can be operably linked to the gene of interest, at or near its 3′ end.

The gene of interest can encode a full length protein or peptide. Alternatively, the gene of interest can encode a truncated, biologically active protein or peptide. The gene can also encode a truncated protein or peptide without biological activity. This protein is useful, for example, as an antigen to produce antibodies. Truncated protein or peptide, in some cases lacks one or more activities associated with the full length protein. In some cases, these truncated proteins can produce dominant negative phenotypes in cells expressing them. Identification of proteins that cause dominant negative phenotypes can be used to characterize biochemical pathways.

It is also understood that vectors that lack a gene of interest, can be operably linked to an endogenous gene or polynucleotide sequence using the methods of the invention, as described herein.

The vectors of the invention can also be used to express polynucleotide sequences for purposes other than producing a protein or peptide. For example, the vectors can be used to express an antisense RNA molecule. Expression of an antisense RNA molecule can be useful to inhibit production of a protein or peptide in a cell expressing the sense RNA. Use of antisense RNA as a research reagent and a therapeutic agent has been described extensively in the art. In the case of antisense RNA, the multiple promoters can be used alone (i.e., the promoter need not contain an unpaired splice donor or exon).

The vectors of the invention can also be used to express ribozymes and other types of enzymatic RNA molecules. Ribozymes are RNA molecules capable of cleaving RNA molecules in a sequence specific hydrolysis reaction. Uses of ribozymes as research reagents and therapeutic agents, particularly in gene therapy applications, has been described extensively in the art.

The vectors can also be used to express structural RNA molecules, useful as research or diagnostic reagents, as probes, or as therapeutic agents.

These vectors, and any of the vectors disclosed herein, and obvious variants recognized by one of ordinary skill in the art, can be used in any of the methods described herein to form any of the compositions producible by those methods.

Methods for Making and Using Cells containing Multi-Promoter/Exon Vectors

The invention encompasses cells containing the vector constructs, cells in which the vector constructs have integrated, and cells which are over-expressing desired gene products.

The methods can be carried out in any cell of eukaryotic origin, including but not limited to mammalian cells (such as rat, mouse, bovine, porcine, sheep, goat, and human), avian cells, fish cells, amphibian cells, reptilian cells, plant cells, and yeast cells. Preferred embodiments include vertebrates and particularly mammals, and more particularly, humans. Examples of useful vertebrate tissues from which cells can be isolated and activated include, but are not limited to, liver, kidney, spleen, bone marrow, thymus, heart, muscle, lung, brain, immune system (including lymphatic), testes, ovary, islet, intestinal, stomach, bone marrow, skin, bone, gall bladder, prostate, bladder, zygotes, embryos, and hematopoietic tissue. Useful vertebrate cell types include, but are not limited to, fibroblasts, epithelial cells, neuronal cells, germ cells (i.e., spermatocytes/spermatozoa and oocytes), stem cells, and follicular cells. Examples of plant tissues from which cells can be isolated and activated include, but are not limited to, leaf tissue, ovary tissue, stamen tissue, pistil tissue, root tissue, tubers, gametes, seeds, embryos, and the like. One of ordinary skill will appreciate, however, that any eukaryotic cell or cell type can be used to activate gene expression using the present invention.

In several embodiments of the invention, overexpression of an endogenous gene or gene product from a particular species is accomplished by activating gene expression in a cell from that species. For example, to overexpress endogenous human proteins, human cells are used. Similarly, to overexpress endogenous bovine proteins, for example bovine growth hormone, bovine cells are used.

The construct can be introduced into primary, secondary, or immortalized cells. Primary cells are cells that have been isolated from a vertebrate and have not been passaged. Secondary cells are primary cells that have been passaged, but are not immortalized. Immortalized cells are cell lines that can be passaged, apparently indefinitely.

In preferred embodiments, the cells are immortalized cell lines. Examples of immortalized cell lines include, but are not limited to, HT1080, HeLa, Jurkat, 293 cells, KB carcinoma, T84 colonic epithelial cell line, Raji, Hep G2 or Hep 3B hepatoma cell lines, A2058 melanoma, U937 lymphoma, and WI38 fibroblast cell line, somatic cell hybrids, and hybridomas.

Any of the cells produced by any of the methods described are useful for screening for expression of a desired gene product and for providing desired amounts of a gene product that is over-expressed in the cell. The cells can be isolated and cloned.

Cells expressing genes from the vector can be used to produce protein in vitro (e.g., for use as a protein therapeutic) or in vivo (e.g., for use in cell therapy).

Any of the cells described herein can be cultured under conditions favoring the production of a gene. As used herein the phrases “conditions favoring the production” of an expression product, “conditions favoring the overexpression” of a gene, and “conditions favoring the activation” of a gene, in a cell or by a cell in vitro refer to any and all suitable environmental, physical, nutritional or biochemical parameters that allow, facilitate, or promote production of an expression product, or overexpression or activation of a gene, by a cell in vitro. Such conditions include the use of culture media, incubation, lighting, humidity, etc., that are optimal or that allow, facilitate, or promote production of an expression product, or overexpression or activation of a gene, by a cell in vitro. Analogously, as used herein the phrases “conditions favoring the production” of an expression product, “conditions favoring the overexpression” of a gene, and “conditions favoring the activation” of a gene, in a cell or by a cell in vivo refer to any and all suitable environmental, physical, nutritional, biochemical, behavioral, genetic, and emotional parameters under which an animal containing a cell is maintained, that allow, facilitate, or promote production of an expression product, or overexpression or activation of a gene, by a cell in a eukaryote in vivo. Whether a given set of conditions are favorable for gene expression, activation, or overexpression, in vitro or in vivo, can be determined by one of ordinary skill using the screening methods described and exemplified below, or other methods for measuring gene expression, activation, or overexpression that are routine in the art.

Commercial growth and production conditions often vary from the conditions used to grow and prepare cells for analytical use (e.g., cloning, protein or nucleic acid sequencing, raising antibodies, X-ray crystallography analysis, enzymatic analysis, and the like). Scale up of cells for growth in roller bottles involves increase in the surface area on which cells can attach. Microcarrier beads are, therefore, often added to increase the surface area for commercial growth. Scale up of cells in spinner culture may involve large increases in volume. Five liters or greater can be required for both microcarrier and spinner growth. Depending on the inherent potency (specific activity) of the protein of interest, the volume can be as low as 1-10 liters. 10-15 liters is more common. However, up to 50-100 liters may be necessary and volume can be as high as 10,000-15,000 liters. In some cases, higher volumes may be required. Cells can also be grown in large numbers of T flasks, for example 50-100.

Despite growth conditions, protein purification on a commercial scale can also vary considerably from purification for analytic purposes. Protein purification in a commercial practical context can be initially the mass equivalent of 10 liters of cells at approximately 10⁴ cells/ml. Cell mass equivalent to begin protein purification can also be as high as 10 liters of cells at up to 10⁶ or 10⁷ cells/ml. As one of ordinary skill will appreciate, however, a higher or lower initial cell mass equivalent can also be advantageously used in the present methods.

Another commercial growth condition, especially when the ultimate product is used clinically, is cell growth in serum-free medium, by which is intended medium containing no serum or not in amounts that are required for cell growth. This obviously avoids the undesired co-purification of toxic contaminants (e.g., viruses) or other types of contaminants, for example, proteins that would complicate purification. Serum-free media for growth of cells, commercial sources for such media, and methods for cultivation of cells in serum-free media, are well-known to those of ordinary skill in the art.

A single cell made by the methods described above can over-express a single gene or more than one gene. For example, more than one gene can be activated by the integration of a single construct or by the integration of multiple constructs in the same cell (i.e., more than one type of construct). Alternatively, multiple vectors, each containing a different gene, can be introduced into the same cell. Therefore, a cell can contain only one type of vector construct or different types of constructs, each capable of activating an endogenous gene, or otherwise expressing a gene of interest.

The invention is also directed to methods for making the cells. For example, the invention encompasses cells expressing an endogenous gene as a result of integration of the vector into its genome by homologous, nonhomologous, or site-specific recombination. The invention also encompasses cells expressing an exogenous gene (e.g. either stably or transiently) from the vector (e.g. either integrated or episomal).

The term “transfection” has been used herein for convenience when discussing introducing a polynucleotide into a cell. However, it is to be understood that the specific use of this term has been applied to generally refer to the introduction of the polynucleotide into a cell and is also intended to refer to the introduction by other methods described herein such as electroporation, liposome-mediated introduction, retrovirus-mediated introduction, and the like (as well as according to its own specific meaning).

The vector can be introduced into the cell by a number of methods known in the art. These include, but are not limited to, electroporation, calcium phosphate precipitation, DEAE dextran, lipofection, and receptor mediated endocytosis, polybrene, particle bombardment, and microinjection. Alternatively, the vector can be delivered to the cell as a viral particle (either replication competent or deficient). Examples of viruses useful for the delivery of nucleic acid include, but are not limited to, adenoviruses, adeno-associated viruses, retroviruses, Herpes viruseses, and vaccinia viruses. Other viruses suitable for delivery of nucleic acid molecules into cells that are known to one of ordinary skill may be equivalently used in the present methods.

Following transfection, the cells are cultured under conditions, as known in the art, suitable for expressing the protein of interest. In embodiments of the invention involving integration of the vector into the host cell genome, the cells are cultured under conditions suitable for integration by homologous, nonhomologous, or site-specific recombination, as the case may be. The cells can also be cultured under conditions suitable for gene expression from the vector.

The vector construct can be introduced into cells on a single DNA construct or on separate constructs and allowed to concatemerize.

The vector can be comprised of double-stranded DNA, single-stranded DNA, combinations of single- and double-stranded DNA, single-stranded RNA, double-stranded RNA, and combinations of single- and double-stranded RNA. Thus, for example, the vector construct could be single-stranded RNA which is converted to cDNA by reverse transcriptase; the cDNA converted to double-stranded DNA; and the double-stranded DNA ultimately recombining with the host cell genome.

In several embodiments of the invention, the constructs are linearized prior to introduction into the cell. Linearization of the expression construct creates free DNA ends capable of reacting with chromosomal ends during the integration process. In embodiments related to activating endogenous genes by nonhomologous recombination, the construct is linearized downstream of the multi-promoter/exon units. In embodiments involving activation of endogenous genes by homologous recombination, the vectors can be linearized downstream of the multi-promoter/exons and targeting sequence. Other suitable linearization sites known to those skilled in the art can also be used.

Vectors containing a gene of interest can also be linearized to promote integration into a host cell genome. Preferably, the vector is linearized outside the multi-promoter/exon/gene of interest transcription unit, and if present, outside other important genetic elements on the vector.

Linearization can be facilitated by, for example, placing a unique restriction site downstream of the regulatory sequences and treating the construct with the corresponding restriction enzyme prior to transfection. While not required, for some applications, such as nonhomologous integration of the vector into the host cell genome), it is advantageous to place a “spacer” sequence between the linearization site and the proximal most functional element (e.g., the unpaired splice donor site) on the construct. When present, the spacer sequence protects the important functional elements on the vector from exonucleolytic degradation during the transfection process. The spacer can be composed of any nucleotide sequence that does not change the essential functions of the vector as described herein.

Circular constructs can also be used to express the gene of interest as an episome. Circular vectors can also be used to integrate exogenous genes into the genome of a host cell, or to activate expression of an endogenous gene by homologous, nonhomologous, or site specific recombination.

The invention also encompasses libraries of cells made by the above methods. A library can encompass all of the clones from a single transfection experiment or a subset of clones from a single transfection experiment. The subset can over-express the same gene or more than one gene, for example, a class of genes. The transfection can have been done with a single type of construct or with more than one type of construct.

A library can also be formed by combining all of the recombinant cells from two or more transfection experiments, by combining one or more subsets of cells from a single transfection experiment or by combining subsets of cells from separate transfection experiments. The resulting library can express the same gene, or more than one gene, for example, a class of genes. Again, in each of these individual transfections, a unique construct or more than one construct can be used.

Libraries can be formed from the same cell type or different cell types.

The library can be composed of a single type of cell containing a single or multiple types of expression constructs. Alternatively, the library can be composed of multiple types of cells containing a single or multiple constructs.

The invention is also directed to methods of using libraries of cells to over-express a gene. The library is screened for the expression of the gene and cells are selected that express the desired gene product. The cell can then be used to purify the gene product for subsequent use. Expression of the cell can occur by culturing the cell in vitro or by allowing the cell to express the gene in vivo.

The invention is also directed to methods of using libraries to identify novel gene and gene products.

Screening

The vectors and methods of the invention can be used to create protein expression libraries. Depending on the characteristics of the protein(s) of interest (e.g., secreted versus intracellular proteins) and the nature of the expression construct used to create the library, any or all of the assays described below can be utilized. Other assay formats can also be used.

ELISA.

Expressed proteins can be detected using the enzyme-linked immunosorbent assay (ELISA). If the expressed gene product is secreted, culture supernatants from pools of activation library cells are incubated in wells containing bound antibody specific for the protein of interest. If a cell or group of cells has activated the gene of interest, then the protein will be secreted into the culture media. By screening pools of library clones (the pools can be from 1 to greater than 100,000 library members), pools containing a cell(s) that has activated the gene of interest can be identified. The cell of interest can then be purified away from the other library members by sib selection, limiting dilution, or other techniques known in the art. In addition to secreted proteins, ELISA can be used to screen for cells expressing intracellular and membrane-bound proteins. In these cases, instead of screening culture supernatants, a small number of cells is removed from the library pool (each cell is represented at least 100-1000 times in each pool), lysed, clarified, and added to the antibody-coated wells. Wells with positive color development contain cells expressing the gene of interest.

ELISA Spot Assay.

ELISA spot are coated with antibodies specific for the protein of interest. Following coating, the wells are blocked with 1% BSA/PBS for 1 hour at 37° C. Following blocking, 100,000 to 500,000 cells from the random activation library are applied to each well (representing 10% of the total pool). In general, one pool is applied to each well. If the frequency of a cell expressing the protein of interest is 1 in 10,000 (i.e., the pool consists of 10,000 individual clones, one of which expresses the protein of interest), then plating 500,000 cells per well will yield 50 specific cells. Cells are incubated in the wells at 37° C. for 24 to 48 hours without being moved or disturbed. At the end of the incubation, the cells are removed and the plate is washed 3 times with PBS/0.05% Tween 20 and 3 times with PBS/1%BSA. Secondary antibodies are applied to the wells at the appropriate concentration and incubated for 2 hours at room temperature or 16 hours at 4° C. These antibodies can be biotinylated or labeled directly with horseradish peroxidase (HRP). The secondary antibodies are removed and the plate is washed with PBS/1% BSA. The tertiary antibody or streptavidin labeled with HRP is added and incubated for 1 hour at room temperature. Wells with spot development contain cells expressing the gene of interest.

FACS assay.

The fluorescence-activated cell sorter (FACS) can be used to screen the random activation library in a number of ways. If the gene of interest encodes a cell surface protein, then fluorescently-labeled antibodies or ligands can be incubated with cells from the activation library. If the gene of interest encodes a secreted protein, then cells can be biotinylated and incubated with streptavidin conjugated to an antibody specific to the protein of interest (Manz et al. (1995) Proc. Natl. Acad. Sci. (USA) 92:1921). Following incubation, the cells are placed in a high concentration of gelatin (or other polymer such as agarose or methylcellulose) to limit diffusion of the secreted protein. As protein is secreted by the cell, it is captured by the antibody bound to the cell surface. The presence of the protein of interest is then detected by a second antibody which is fluorescently labeled. For both secreted and membrane bound proteins, the cells can then be sorted according to their fluorescence signal. Fluorescent cells can then be isolated, expanded, and further enriched by FACS, limiting dilution, or other cell purification techniques known in the art.

Magnetic Bead Separation.

The principle of this technique is similar to FACS. Membrane bound proteins and captured secreted proteins (as described above) are detected by cells from the library with an antibody-conjugated magnetic beads that are specific for the protein of interest. If the protein is present on the surface of a cell, the magnetic beads will bind to that cell. Using a magnet, the cells expressing the protein of interest can be purified away from the other cells in the library. The cells are then released from the beads, expanded, analyzed, and further purified if necessary.

RT-PCR.

A small number of cells (equivalent to at least the number of individual clones in the pool) is harvested and lysed to allow purification of the RNA. Following isolation, the RNA is reversed-transcribed using reverse transcriptase. PCR is then carried out using primers specific for the cDNA of the gene of interest.

Alternatively, primers can be used that span the synthetic exon in the expression construct and the exon of the endogenous gene. This primer will not hybridize to and amplify the endogenously expressed gene of interest. Conversely, if the expression construct has integrated upstream of the gene of interest and activated gene expression, then this primer, in conjunction with a second primer specific for the gene will amplify the activated gene by virtue of the presence of the synthetic exon spliced onto the exon from the endogenous gene. Thus, this method can be used to detect activated genes in cells that normally express the gene of interest at lower than desired levels.

Phenotypic Selection.

In this embodiment, cells can be selected based on a phenotype conferred by the activated gene. Examples of phenotypes that can be selected for include proliferation, growth factor independent growth, colony formation, cellular differentiation (e.g., differentiation into a neuronal cell, muscle cell, epithelial cell, etc.), anchorage independent growth, activation of cellular factors (e.g., kinases, transcription factors, nucleases, etc.), gain or loss of cell-cell adhesion, migration, and cellular activation (e.g., resting versus activated T cells). Isolation of activated cells demonstrating a phenotype, such as those described above, is important because the activation of an endogenous gene by the integrated construct is presumably responsible for the observed cellular phenotype. Thus, the activated gene may be an important therapeutic drug or drug target for treating or inducing the observed phenotype.

The sensitivity of each of the above assays can be effectively increased by transiently upregulating gene expression in the library cells. This can be accomplished for NF-κB site-containing promoters (on the expression construct) by adding PMA and tumor necrosis factor-α, e.g., to the library. Separately, or in conjunction with PMA and TNF-α, sodium butyrate can be added to further enhance gene expression. Addition of these reagents can increase expression of the protein of interest, thereby allowing a lower sensitivity assay to be used to identify the cell of interest.

Since large expression libraries are created to maximize expression of many genes, it is advantageous to organize the library clones in pools. Each pool can consist of 1 to greater than 100,000 individual clones. Thus, in a given pool, many proteins are produced, often in dilute concentrations (due to the overall size of the pool and the limited number of cells within the pool that produce a given protein). Thus, concentration of the proteins prior to screening effectively increases the ability to detect the expressed proteins in the screening assay. One particularly useful method of concentration is ultrafiltration; however, other methods can also be used. For example, proteins can be concentrated non-specifically, or semi-specifically by adsorption onto ion exchange, hydrophobic, dye, hydroxyapatite, lectin, and other suitable resins under conditions that bind most or all proteins present. The bound proteins can then be removed in a small volume prior to screening. It is advantageous to grow the cells in serum free media to facilitate the concentration of proteins.

In another embodiment, a useful sequence that can be included on the expression construct is an epitope tag. The epitope tag can consist of an amino acid sequence that allows affinity purification of the expressed protein (e.g., on immunoaffinity or chelating matrices). Thus, by including an epitope tag on the expression construct, all of the expressed proteins from a library can be purified. By purifying the activated proteins away from other cellular and media proteins, screening for novel proteins and enzyme activities can be facilitated. In some instances, it may be desirable to remove the epitope tag following purification of the activated protein. This can be accomplished by including a protease recognition sequence (e.g., Factor IIa or enterokinase cleavage site) downstream from the epitope tag on the expression construct. Incubation of the purified, activated protein(s) with the appropriate protease will release the epitope tag from the proteins(s).

In libraries in which an epitope tag sequence is included on the vector construct, all of the expressed proteins can be purified away from all other cellular and media proteins using affinity purification. This not only concentrates the expressed proteins, but also purifies them away from other activities that can interfere with the assay used to screen the library.

Once a pool of clones containing cells over-expressing the gene of interest is identified, steps can be taken to isolate the expressing cell. Isolation of the cell can be accomplished by a variety of methods known in the art. Examples of cell purification methods include limiting dilution, fluorescence activated cell sorting, magnetic bead separation, sib selection, and single colony purification using cloning rings.

In preferred embodiments of the invention, the methods include a process wherein the expression product is purified. In highly preferred embodiments, the cells expressing gene of interest are cultured so as to produce amounts of gene product feasible for commercial application, and especially diagnostic and therapeutic and drug discovery uses.

In Vivo Protein Production

Cells of the present invention are useful, as populations of recombinant cell lines, as populations of recombinant primary or secondary cells, recombinant clonal cell strains or lines, recombinant heterogeneous cell strains or lines, and as cell mixtures in which at least one representative cell of one of the four preceding categories of recombinant cells is present. Such cells can be used in a delivery system for treating an individual with an abnormal or undesirable condition which responds to delivery of a therapeutic product, which is either: 1) a therapeutic protein (e.g., a protein which is absent, underproduced relative to the individual's physiologic needs, defective or inefficiently or inappropriately utilized in the individual; a protein with novel functions, such as enzymatic or transport functions) or 2) a therapeutic nucleic acid (e.g., RNA which inhibits gene expression or has intrinsic enzymatic activity). In the method of the present invention of providing a therapeutic protein or nucleic acid, recombinant primary cells, clonal cell strains or heterogeneous cell strains are administered to an individual in whom the abnormal or undesirable condition is to be treated or prevented, in sufficient quantity and by an appropriate route, to express or make available the protein or exogenous DNA at physiologically relevant levels. A physiologically relevant level is one which either approximates the level at which the product is normally produced in the body or results in improvement of the abnormal or undesirable condition. According to an embodiment of the invention described herein, the recombinant immortalized cell lines to be administered can be enclosed in one or more semipermeable barrier devices. The permeability properties of the device are such that the cells are prevented from leaving the device upon implantation into an animal, but the therapeutic product is freely permeable and can leave the barrier device and enter the local space surrounding the implant or enter the systemic circulation. For example, hGH, HEPO, human insulinotropin, hGM-CSF, hG-CSF, human.alpha.-interferon, or human FSH-beta. can be delivered systemically in humans for therapeutic benefits.

Barrier devices are particularly useful and allow recombinant immortalized cells, recombinant cells from another species (recombinant xenogeneic cells), or cells from a nonhistocompatibility-matched donor (recombinant allogeneic cells) to be implanted for treatment of human or animal conditions or for agricultural uses (i.e., meat and dairy production). Barrier devices also allow convenient short-term (i.e., transient) therapy by providing ready access to the cells for removal when the treatment regimen is to be halted for any reason.

A number of synthetic, semisynthetic, or natural filtration membranes can be used for this purpose, including, but not limited to, cellulose, cellulose acetate, nitrocellulose, polysulfone, polyvinylidene difluoride, polyvinyl chloride polymers and polymers of polyvinyl chloride derivatives. Barrier devices can be utilized to allow primary, secondary, or immortalized cells from another species to be used for gene therapy in humans.

In Vitro Protein Production

Recombinant cells from human or non-human species according to this invention can also be used for in vitro protein production. The cells are maintained under conditions, as are known in the art, which result in expression of the protein. Proteins expressed using the methods described can be purified from cell lysates or cell supernatants in order to purify the desired protein. Proteins made according to this method include therapeutic proteins that can be delivered to a human or non-human animal by conventional pharmaceutical routes as is known in the art (e.g., oral, intravenous, intramuscular, intranasal or subcutaneous). Such proteins include hGH, hEPO, and human insulinotropin, hGM-CSF, hG-CSF, FSH-beta. or alpha-interferon. These cells can be immortalized, primary, or secondary cells. The use of cells from other species may be desirable in cases where the non-human cells are advantageous for protein production purposes where the non-human protein is therapeutically or commercially useful, for example, the use of cells derived from salmon for the production of salmon calcitonin, the use of cells derived from pigs for the production of porcine insulin, and the use of bovine cells for the production of bovine growth hormone.

Drug Screening

The cells expressing proteins by the present invention can be used to identify novel drugs, to characterize existing drugs, or to improve existing drugs. Accordingly, cells produced by the methods of the invention can be formatted to allow high through-put screening. For example, the cells can be modified to express a reporter gene in response to activation or inhibition of the protein expressed from the vector. The cells can also be modified to express other proteins, in addition to the protein expressed from the vector of the invention, to allow detection of agonists and antagonists. For example, to identify drug compounds that act upon a GPCR, the cell can be modified to express a suitable G protein capable of signal transduction via the GPCR of interest.

The cells of the invention can be treated with compounds to identify compounds that cause a particular cellular or biochemical response. The number of compounds tested can range from 1 to 100,000 or more.

Useful assays for high through put drug screening have been described for ion channels, GPCRs, enzymes, and other proteins and peptides, etc. (16-22). Other assays known to those skilled in the art can also be used.

Proteins produced by the cells of the invention can also be used to identify drug compounds in cell free assays.

Proteins

The invention encompasses over-expression of genes both in vivo and in vitro. Therefore, the cells could be used in vitro to produce desired amounts of a gene product or could be used in vivo to provide that gene product in the intact animal.

The invention also encompasses the proteins produced by the methods described herein. The proteins can be produced from either known, or previously unknown genes. Examples of known proteins that can be produced by this method include, but are not limited to, erythropoietin, insulin, growth hormone, glucocerebrosidase, tissue plasminogen activator, granulocyte-colony stimulating factor, granulocyte/macrophage colony stimulating factor, interferon α, interferon β, interferon γ, interleukin-2, interleukin-6, interleukin-11, interleukin-12, TGF β, blood clotting factor V, blood clotting factor VII, blood clotting factor VIII, blood clotting factor IX, blood clotting factor X, TSH β, bone growth factor 2, bone growth factor-7, tumor necrosis factor, alpha-1 antitrypsin, anti-thrombin III, leukemia inhibitory factor, glucagon, Protein C, protein kinase C, macrophage colony stimulating factor, stem cell factor, follicle stimulating hormone β, urokinase, nerve growth factors, insulin-like growth factors, insulinotropin, parathyroid hormone, lactoferrin, complement inhibitors, platelet derived growth factor, keratinocyte growth factor, neurotropin-3, thrombopoietin, chorionic gonadotropin, thrombomodulin, alpha glucosidase, epidermal growth factor, FGF, macrophage-colony stimulating factor, and cell surface receptors for each of the above-described proteins, cholinergic receptors, GABA receptors, ion channels, G protein coupled receptors, and other medically relevant proteins.

Where the protein product from the expressing cell is purified, any method of protein purification known in the art can be employed.

### EXAMPLE 1

### Expression of Proteins Using Multi-promoter Vectors by Non-homologous Recombination

The vectors of the present invention can be used to activate protein expression from endogenous genes using nonhomologous recombination. Protein expression is achieved by integrating the vectors randomly or semi-randomly throughout the genome of a host cell. When the vector integrates into or upstream of an endogenous gene, the multiple promoter/exons on the vector will drive expression of the operably linked endogenous gene. As a result, the vectors of the present invention can be used to achieve higher levels of expression without the need for gene amplification. Alternatively, the vectors of the invention can be used in conjunction with gene amplification to achieve higher levels of expression with fewer amplification steps or higher levels of expression overall.

Methods for activating endogenous genes by nonhomologous recombination have been described (U.S. patent application Ser. No. 09/276,820, incorporated herein by reference for such methods). These previously described methods can be used to activate endogenous genes with the vectors of the present invention.

One of the advantages of the activating endogenous genes using nonhomologous recombination is that virtually any gene can be expressed. However, since genes have different genomic structures, including different intron/exon boundaries and locations of start codons, multiple vectors containing activation exons with different coding information can be used to activate the maximum number of different genes within a population of cells. As discussed above, the activation exons in different vectors can contain: a translation site in different reading frames, signal peptides, partial signal peptides, epitope tags, or other sequences.

These constructs can be transfected separately into cells to produce libraries. Each library contains cells with a unique set of activated genes. Some genes will be activated by several different expression constructs. In addition, portions of a gene can be activated to produce truncated, biologically active proteins. Truncated proteins can be produced, for example, by integration of an expression construct into introns or exons in the middle of an endogenous gene rather than upstream of the second exon.

Nonhomologous integration of the construct into the genome of a cell results in the operable linkage between the regulatory elements from the vector and the exons from an endogenous gene. In preferred embodiments, the insertion of the vector regulatory sequences is used to upregulate expression of the endogenous gene. Upregulation of gene expression includes converting a transcriptionally silent gene to a transcriptionally active gene. It also includes enhancement of gene expression for genes that are already transcriptionally active, but produce protein at levels lower than desired. In other embodiments, expression of the endogenous gene can be affected in other ways such as downregulation of expression, creation of an inducible phenotype, or changing the tissue specificity of expression.

According to the invention, in vitro methods of production of a gene expression product comprise, for example, (a) introducing a vector of the invention into a cell; (b) allowing the vector to integrate into the genome of the cell by non-homologous recombination; (c) allowing over-expression of an endogenous gene in the cell by upregulation of the gene by the transcriptional regulatory sequences contained on the vector; (d) screening the cell for over-expression of the endogenous gene; and (e) culturing the cell under conditions favoring the production of the expression product of the endogenous gene by the cell. Such in vitro methods of the invention can further comprise isolating the expression product to produce an isolated gene expression product. In such methods, any art-known method of protein isolation can be advantageously used, including but not limited to chromatography (e.g., HPLC, FPLC, LC, ion exchange, affinity, size exclusion, and the like), precipitation (e.g., ammonium sulfate precipitation, immunoprecipitation, and the like), electrophoresis, and other methods of protein isolation and purification that will be familiar to one of ordinary skill in the art.

Analogously, in vivo methods of production of a gene expression product can comprise, for example, (a) introducing a vector of the invention into a cell; (b) allowing the vector to integrate into the genome of the cell by non-homologous recombination; (c) allowing over-expression of an endogenous gene in the cell by upregulation of the gene by the transcriptional regulatory sequence contained on the vector; (d) screening the cell for over-expression of the endogenous gene; and (e) introducing the isolated and cloned cell into a eukaryote under conditions favoring the overexpression of the endogenous gene by the cell in vivo in the eukaryote. According to this aspect of the invention, any eukaryote can be advantageously used, including fungi (particularly yeasts), plants, and animals, more preferably animals, still more preferably vertebrates, and most preferably mammals, particularly humans. In certain related embodiments, the invention provides such methods which further comprise isolating and cloning the cell prior to introducing it into the eukaryote.

As used herein, the phrase “activating an endogenous gene” means inducing the production of a transcript encoding the endogenous gene at levels higher than those normally found in the cell containing the endogenous gene. In some applications, “activating an endogenous gene” can also mean producing the protein, or a portion of the protein, encoded by the endogenous gene at levels higher than those normally found in the cell containing the endogenous gene.

The invention is also directed to methods for making the cells described above by one or more of the following: introducing one or more of the vector constructs; allowing the introduced construct(s) to integrate into the genome of the cell by non-homologous recombination; allowing over-expression of one or more endogenous genes in the cell; and isolating and cloning the cell.

Following transfection, the cells are cultured under conditions, as known in the art, suitable for nonhomologous integration between the vector and the host cell's genome. Cells containing the nonhomologously integrated vector can be further cultured under conditions, as known in the art, allowing expression of activated endogenous genes.

The vector construct can be introduced into cells on a single DNA construct or on separate constructs and allowed to concatemerize.

The vector construct can be a double-stranded DNA vector construct, vector constructs also include single-stranded DNA, combinations of single- and double-stranded DNA, single-stranded RNA, double-stranded RNA, and combinations of single- and double-stranded RNA. Thus, for example, the vector construct could be single-stranded RNA which is converted to cDNA by reverse transcriptase, the cDNA converted to double-stranded DNA, and the double-stranded DNA ultimately recombining with the host cell genome.

The vector can include selectable markers and amplifiable markers, as described above in the Detailed Description of the Invention sections entitled “Selectable Markers” and “Amplifiable Markers.” Cells, therefore, can be selected with agents that permit the survival of cells containing a single or multiple copies of the integrated vector, as described herein.

In preferred embodiments, the constructs are linearized prior to introduction into the cell. Linearization of the expression construct creates free DNA ends capable of reacting with chromosomal ends during the integration process. In general, the construct is linearized downstream of the 3′ most promoter/exon unit. Linearization can be facilitated by, for example, placing a unique restriction site downstream of the regulatory sequences and treating the construct with the corresponding restriction enzyme prior to transfection. While not required, it is advantageous to place a “spacer” sequence between the linearization site and the proximal most functional element (e.g., the unpaired splice donor site) on the construct. When present, the spacer sequence protects the important functional elements on the vector from exonucleolytic degradation during the transfection process. The spacer can be composed of any nucleotide sequence that does not change the essential functions of the vector as described herein.

Circular constructs can also be used to activate endogenous gene expression. It is known in the art that circular plasmids, upon transfection into cells, can integrate into the host cell genome. Presumably, DNA breaks occur in the circular plasmid during the transfection process, thereby generating free DNA ends capable of joining to chromosome ends. Some of these breaks in the construct will occur in a location that does not destroy essential vector functions (e.g., the break will occur downstream of the regulatory sequence), and therefore, will allow the construct to be integrated into a chromosome in a configuration capable of activating an endogenous gene. As described above, spacer sequences can be placed on the construct (e.g., downstream of the regulatory sequences). During transfection, breaks that occur in the spacer region will create free ends at a site in the construct suitable for activation of an endogenous gene following integration into the host cell genome.

The invention also encompasses libraries of cells made by the above described methods. A library can encompass all of the clones from a single transfection experiment or a subset of clones from a single transfection experiment. The subset can over-express the same gene or more than one gene, for example, a class of genes. The transfection can have been done with a single type of construct or with more than one type of construct.

A library can also be formed by combining all of the recombinant cells from two or more transfection experiments, by combining one or more subsets of cells from a single transfection experiment or by combining subsets of cells from separate transfection experiments. The resulting library can express the same gene, or more than one gene, for example, a class of genes. Again, in each of these individual transfections, a unique construct or more than one construct can be used.

Libraries can be formed from the same cell type or different cell types.

The library can be composed of a single type of cell containing a single type of expression construct which has been integrated into chromosomes at spontaneous DNA breaks or at breaks generated by radiation, restriction enzymes, and/or DNA breaking agents, applied either together (to the same cells) or separately (applied to individual groups of cells and then combining the cells together to produce the library). The library can be composed of multiple types of cells containing a single or multiple constructs which were integrated into the genome of a cell treated with radiation, restriction enzymes, and/or DNA breaking agents, applied either together (to the same cells) or separately (applied to individual groups of cells and then combining the cells together to produce the library).

The invention is also directed to methods for making libraries by selecting various subsets of cells from the same or different transfection experiments. For example, all of the cells expressing nuclear factors (as determined by the presence of nuclear green fluorescent protein in cells transfected with construct 20) can be pooled to create a library of cells with activated nuclear factors. Similarly, cells expressing membrane or secreted proteins can be pooled. Cells can also be grouped by phenotype, for example, growth factor independent growth, growth factor independent proliferation, colony formation, cellular differentiation (e.g., differentiation into a neuronal cell, muscle cell, epithelial cell, etc.), anchorage independent growth, activation of cellular factors (e.g., kinases, transcription factors, nucleases, etc.), gain or loss of cell-cell adhesion, migration, or cellular activation (e.g., resting versus activated T cells).

The invention is also directed to methods of using libraries of cells to over-express an endogenous gene. The library is screened for the expression of the gene and cells are selected that express the desired gene product. The cell can then be used to purify the gene product for subsequent use. Expression of the cell can occur by culturing the cell in vitro or by allowing the cell to express the gene in vivo.

The invention is also directed to methods of using libraries to identify novel gene and gene products.

The invention is also directed to methods for increasing the efficiency of gene activation by treating the cells with agents that stimulate or effect the patterns of nonhomologous integration. The methods of the invention can include introducing double strand breaks into the DNA of the cell containing the endogenous gene to be over-expressed. These methods introduce double-strand breaks into the genomic DNA in the cell prior to or simultaneously with vector integration. The mechanism of DNA breakage can have a significant effect on the pattern of DNA breaks in the genome. As a result, DNA breaks produced spontaneously or artificially with radiation, restriction enzymes, bleomycin, or other breaking agents, can occur in different locations.

The invention is also directed to non-human transgenic animals. The genetically engineered host cells can be used to produce such animals. In this embodiment, the nucleic acid constructs of the present invention are integrated into the genome of a cell from which a transgenic animal develops and which remains in the genome of the mature animal in one or more cell types or tissues of the transgenic animal. In one example, an inducible promoter is used such that the gene of interest is expressed in a specific tissue in the transgenic animal, for example, expressed in the mammary gland so that the protein of interest can be purified from milk. The animal is produced by introducing nucleic acid into the male pronuclei of a fertilized oocyte by well-known methods.

### EXAMPLE 2

### Activation of Endogenous Genes by Homologous Recombination

The vectors of the present invention can be used to activate protein expression from endogenous genes using homologous recombination. Protein expression is achieved by integrating the vectors into or upstream of an endogenous gene in a site-specific fashion. Accordingly, in this embodiment of the invention, the multipromoter/exon vectors contain one or more targeting sequences. Following homologous recombination with the genome and operable linkage to an endogenous gene, the multiple promoter/exons on the vector will drive expression of the operably linked endogenous gene. As a result, the vectors of the present invention can be used to achieve higher levels of expression without the need for gene amplification. Alternatively, the vectors of the invention can be used in conjunction with gene amplification to achieve higher levels of expression with fewer amplification steps, or higher levels of expression overall.

Methods for activating endogenous genes by homologous recombination have been described (2-6, incorporated herein by reference for these methods). These previously described methods can be used to activate endogenous genes with the vectors of the present invention.

While methods for activating endogenous genes are incorporated herein by reference, the vectors and methods of the present invention are discussed below in context of previously described methods.

The DNA Construct

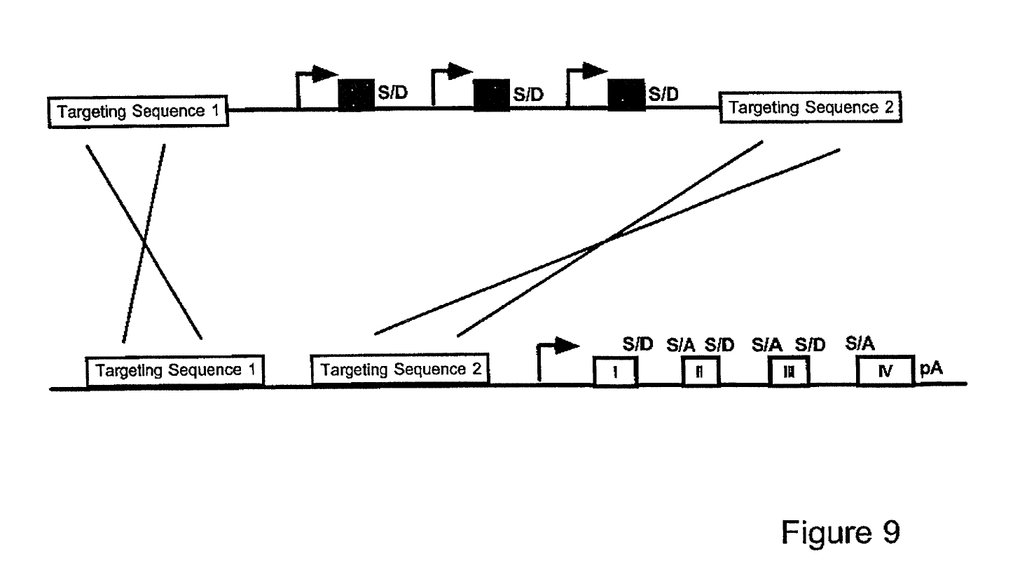

The DNA construct of the present embodiment includes at least the following components: a targeting sequence and two or more promoter/exon units. An example of a DNA vector useful in this embodiment of the invention is shown in FIG. 9. As described herein, additional genetic elements, such as selectable markers or amplifiable markers, can be frequently included on the vector.

The DNA in the construct can be referred to as exogenous. The term “exogenous” is defined herein as DNA which is introduced into a cell by the method of the present invention, such as with the DNA constructs defined herein. Exogenous DNA can possess sequences identical to or different from the endogenous DNA present in the cell prior to transfection.

The Targeting Sequence or Sequences

The targeting sequence or sequences are DNA sequences that permit legitimate homologous recombination into the genome of the selected cell containing the gene of interest. Targeting sequences are, generally, DNA sequences that are homologous to (i.e., identical or sufficiently similar to cellular DNA such that the targeting sequence and cellular DNA can undergo homologous recombination) DNA sequences normally present in the genome of the cells (e.g., coding or noncoding DNA, lying upstream of the transcriptional start site, within, or downstream of the transcriptional stop site of a gene of interest, or sequences present in the genome through a previous modification). The targeting sequence or sequences used are selected with reference to the site into which the DNA in the DNA construct is to be inserted.

One or more targeting sequences can be employed. For example, a circular plasmid or DNA fragment preferably employs a single targeting sequence. A linear plasmid or DNA fragment preferably employs two targeting sequences. A linear sequence or sequences can, independently, be within the gene of interest (such as, the sequences of an exon and/or intron), immediately adjacent to the gene of interest (i.e., with no additional nucleotides between the targeting sequence and the coding region of the gene of interest), upstream gene of interest (such as the sequences of the upstream non-coding region or endogenous promoter sequences), or upstream of and at a distance from the gene (such as, sequences upstream of the endogenous promoter). The targeting sequence or sequences can include those regions of the targeted gene presently known or sequenced and/or regions further upstream which are structurally uncharacterized but can be mapped using restriction enzymes and determined by one skilled in the art.

As taught herein, gene targeting can be used to insert a regulatory sequence isolated from a different gene, assembled from components isolated from difference cellular and/or viral sources, or synthesized as a novel regulatory sequence by genetic engineering methods within, immediately adjacent to, upstream, or at a substantial distance from an endogenous cellular gene. Alternatively or additionally, sequences that affect the structure or stability of the RNA or protein produced can be replaced, removed, added, or otherwise modified by targeting. For example, RNA stability elements, splice sites, and/or leader sequences of RNA molecules can be modified to improve or alter the function, stability, and/or translatability of an RNA molecule. Protein sequences can also be altered, such as signal sequences, propeptide sequences, active sites, and/or structural sequences for enhancing or modifying transport, secretion, or functional properties of a protein. According to this method, introduction of the exogenous DNA results in the alteration of the normal expression properties of a gene and/or the structural properties of a protein or RNA.

The Targeted Gene and Resulting Product

The DNA construct, when transfected into cells, such as primary, secondary or immortalized cells, can control the expression of a desired product for example, the active or, functional portion of the protein or RNA. The product can be, for example, a hormone, a cytokine, an antigen, an antibody, an enzyme, a clotting factor, a transport protein, a receptor, a regulatory protein, a structural protein, a transcription factor, an anti-sense RNA, or a ribozyme. Additionally, the product can be a protein or a nucleic acid which does not occur in nature (i.e., a fusion protein or nucleic acid).

The method as described herein can produce one or more therapeutic products from known genes. Examples of known genes that can be over-expressed in the present embodiment are discussed above.

Selectable Markers and Amplification

The identification of the targeting event can be facilitated by the use of one or more selectable marker genes. These markers can be included in the targeting construct or be present on different constructs. Selectable markers can be divided into two categories: positively selectable and negatively selectable (in other words, markers for either positive selection or negative selection). In positive selection, cells expressing the positively selectable marker are capable of surviving treatment with a selective agent (such as neo, xanthine-guanine phosphoribosyl transferase (gpt), dhfr, adenosine deaminase (ada), puromycin (pac), hygromycin (hyg), CAD which encodes carbamyl phosphate synthase, aspartate transcarbamylase, and dihydro-orotase glutamine synthetase (GS), multidrug resistance 1 (mdrl) and histidine D (hisD), allowing for the selection of cells in which the targeting construct integrated into the host cell genome. In negative selection, cells expressing the negatively selectable marker are destroyed in the presence of the selective agent. The identification of the targeting event can be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker (Mansour et al. (1988) Nature 336:348-352). Markers useful for this purpose include the Herpes Simplex Virus thymidine kinase (TK) gene or the bacterial gpt gene.

A variety of selectable markers can be incorporated into primary, secondary or immortalized cells. For example, a selectable marker which confers a selectable phenotype such as drug resistance, nutritional auxotrophy, resistance to a cytotoxic agent or expression of a surface protein, can be used. Selectable marker genes which can be used include neo, gpt, dhfr, ada, pac, hyg, CAD, GS, mdrl and hisD. The selectable phenotype conferred makes it possible to identify and isolate recipient cells.

Amplifiable genes encoding selectable markers (e.g., ada, GS, dhfr and the multifunctional CAD gene) have the added characteristic that they enable the selection of cells containing amplified copies of the selectable marker inserted into the genome. This feature provides a mechanism for significantly increasing the copy number of an adjacent or linked gene for which amplification is desirable. Mutated versions of these sequences showing improved selection properties and other amplifiable sequences can also be used.

The order of components in the DNA construct can vary. Where the construct is a circular plasmid, the order of elements in the resulting structure can be: targeting sequence—plasmid DNA (comprised of sequences used for the selection and/or replication of the targeting plasmid in a microbial or other suitable host)—selectable marker(s)—promoter/exon units. Preferably, the plasmid containing the targeting sequence and exogenous DNA elements is cleaved with a restriction enzyme that cuts one or more times within the targeting sequence to create a linear or gapped molecule prior to introduction into a recipient cell, such that the free DNA ends increase the frequency of the desired homologous recombination event as described herein. In addition, the free DNA ends can be treated with an exonuclease to create protruding 5′ or 3′ overhanging single-stranded DNA ends to increase the frequency of the desired homologous recombination event. In this embodiment, homologous recombination between the targeting sequence and the cellular target will result in two copies of the targeting sequences, flanking the elements contained within the introduced plasmid.

Where the construct is linear, the order can be, for example: a first targeting sequence—selectable marker-promoter/exon units—a second targeting sequence or, in the alternative, a first targeting sequence-promoter/exon units—DNA encoding a selectable marker—a second targeting sequence. Cells that stably integrate the construct will survive treatment with the selective agent; a subset of the stably transfected cells will be homologously recombinant cells. The homologously recombinant cells can be identified by a variety of techniques, including PCR, Southern hybridization and phenotypic screening.

In another embodiment, the order of the construct can be: a first targeting sequence—selectable marker-promoter/exon units—an intron—a splice-acceptor site—a second targeting sequence.

Alternatively, the order of components in the DNA construct can be, for example: a first targeting sequence—selectable marker 1—promoter/exon units—a second targeting sequence—selectable marker 2, or, alternatively, a first targeting sequence—promoter/exon units—selectable marker 1—a second targeting sequence—selectable marker 2. In this embodiment selectable marker 2 displays the property of negative selection. That is, the gene product of selectable marker 2 can be selected against by growth in an appropriate media formulation containing an agent (typically a drug or metabolite analog) which kills cells expressing selectable marker 2. Recombination between the targeting sequences flanking selectable marker 1 with homologous sequences in the host cell genome results in the targeted integration of selectable marker 1, while selectable marker 2 is not integrated. Such recombination events generate cells that are stably transfected with selectable marker 1 but not stably transfected with selectable marker 2, and such cells can be selected for by growth in the media containing the selective agent that selects for selectable marker 1 and the selective agent that selects against selectable marker 2.

The DNA construct also can include a positively selectable marker that allows for the selection of cells containing amplified copies of that marker. The amplification of such a marker results in the co-amplification of flanking DNA sequences. In this embodiment, the order of construct components is, for example: a first targeting sequence—an amplifiable positively selectable marker—a second selectable marker (optional)—promoter/exon units—a second targeting DNA sequence.

In this embodiment, the activated gene can be further amplified by the inclusion of a selectable marker gene which has the property that cells containing amplified copies of the selectable marker gene can be selected for by culturing the cells in the presence of the appropriate selectable agent. The activated endogenous gene will be amplified in tandem with the amplified selectable marker gene. Cells containing many copies of the activated endogenous gene can produce very high levels of the desired protein and are useful for in vitro protein production and gene therapy.

In any embodiment, the selectable and amplifiable marker genes do not have to lie immediately adjacent to each other.

Optionally, the DNA construct can include a bacterial origin of replication and bacterial antibiotic resistance markers or other selectable markers, which allow for large-scale plasmid propagation in bacteria or any other suitable cloning/host system. A DNA construct which includes DNA encoding a selectable marker, along with additional sequences, such as a promoter, and splice junctions, can be used to confer a selectable phenotype upon transfected cells. Such a DNA construct can be co-transfected into primary or secondary cells, along with a targeting DNA sequence, using methods described herein.

Transfection and Homologous Recombination

According to the present method, the construct is introduced into the cell, such as a primary, secondary, or immortalized cell, as a single DNA construct, or as separate DNA sequences which become incorporated into the chromosomal or nuclear DNA of a transfected cell.

The targeting DNA construct, including the targeting sequences, multipromoter/exon units, and selectable marker gene(s), can be introduced into cells on a single DNA construct or on separate constructs. The total length of the DNA construct will vary according to the number of components (targeting sequences, regulatory sequences, exons, selectable marker gene, and other elements, for example) and the length of each. The entire construct length will generally be at least about 200 nucleotides. Further, the DNA can be introduced as linear, double-stranded (with or without single-stranded regions at one or both ends), single-stranded, or circular.

Any of the construct types of the disclosed invention is then introduced into the cell to obtain a transfected cell. The transfected cell is maintained under conditions that permit homologous recombination, as is known in the art (Capecchi, M. R., (1989) Science 244:1288-1292). When the homologously recombinant cell is maintained under conditions sufficient for transcription of the DNA, the regulatory region introduced by the targeting construct, as in the case of a promoter, will activate transcription.

The DNA constructs can be introduced into cells by a variety of physical or chemical methods, including the transfection methods described above.

Optionally, the targeting DNA can be introduced into a cell in two or more separate DNA fragments. In the event two fragments are used, the two fragments share DNA sequence homology (overlap) at the 3′ end of one fragment and the 5′ end of the other, while one carries a first targeting sequence and the other carries a second targeting sequence. Upon introduction into a cell, the two fragments can undergo homologous recombination to form a single fragment with the first and second targeting sequences flanking the region of overlap between the two original fragments. The product fragment is then in a form suitable for homologous recombination with the cellular target sequences. More than two fragments can be used, designed such that they will undergo homologous recombination with each other to ultimately form a product suitable for homologous recombination with the cellular target sequences as described above.

The Homologously Recombinant Cells

The targeting event results in the insertion of the multi-promoter/exon units of the targeting construct, placing the endogenous gene under their control. Optionally, the targeting event can simultaneously result in the deletion of the endogenous regulatory element, such as the deletion of a tissue-specific negative regulatory element. The targeting event can replace an existing element; for example, a tissue-specific enhancer can be replaced by an enhancer that has broader or different cell-type specificity than the naturally-occurring elements, or displays a pattern of regulation or induction that is different from the corresponding nontransfected cell. In this embodiment the naturally occurring sequences are deleted and new sequences are added. Alternatively, the endogenous regulatory elements are not removed or replaced but are disrupted of disabled by the targeting event, such as by targeting the exogenous sequences within the endogenous regulatory elements.

After the DNA is introduced into the cell, the cell is maintained under conditions appropriate for homologous recombination to occur between the genomic DNA and a portion of the introduced DNA, as is known in the art (Capecchi, M. R. (1989) Science 244:1288-1292).

Homologous recombination between the genomic DNA and the introduced DNA results in a homologously recombinant cell, such as a fungal, plant or animal, and particularly, primary, secondary, or immortalized human or other mammalian cell in which sequences which alter the expression of an endogenous gene are operatively linked to an endogenous gene encoding a product, producing multiple new transcription units with expression and/or coding potential that is different from that of the endogenous gene. Particularly, the invention includes a homologously recombinant cell comprising multiple promoter/exon units, which are introduced at a predetermined site by a targeting DNA construct, and are operatively linked to the second exon of an endogenous gene. The resulting homologously recombinant cells are cultured under conditions that select for amplification, if appropriate, of the DNA encoding the amplifiable marker and the novel transcriptional unit. With or without amplification, cells produced by this method can be cultured under conditions, as are known in the art, suitable for the expression of the protein, thereby producing the protein in vitro, or the cells can be used for in vivo delivery of a therapeutic protein (i.e., gene therapy).

### EXAMPLE 3

### Expression of Genes Cloned into, Inserted into, or Otherwise Combined with Multi-promoter/exon Vectors

The vectors of the present invention can be used to express protein from isolated genomic fragments. Protein expression is achieved combining the vector with a genomic fragment downstream of and in the same orientation as the multipromoter exon units. When the vector containing the genomic fragment is introduced into a suitable cell, the multiple promoter/exons on the vector will drive expression of the operably linked gene. As a result, the vectors of the present invention can be used to achieve higher levels of expression without the need for gene amplification. Alternatively, the vectors of the invention can be used in conjunction with gene amplification to achieve higher levels of expression with fewer amplification steps, or higher levels of expression overall.

Methods for expressing genes by from cloned genomic DNA have been described (U.S. patent application Ser. No. 09/276,820, incorporated herein by reference). These previously described methods can be used to express genes using the vectors of the present invention.

It is recognized that any of the vectors described herein can be integrated into, or otherwise combined with, genomic DNA prior to transfection into a eukaryotic host cell. This permits high level expression from virtually any gene in the genome, regardless of the normal expression characteristics of the gene. Thus, the vectors of the invention can be used to activate expression from genes encoded by isolated genomic DNA fragments. To accomplish this, the vector is integrated into, or otherwise combined with, genomic DNA containing at least one gene, or portion of a gene. Typically, the expression vector must be positioned within or upstream of a gene in order to activate gene expression. Once inserted (or joined), the downstream gene can be expressed (as a transcript or a protein) by introducing the vector/genomic DNA into an appropriate eukaryotic host cell. Following introduction into the host cell, the vector encoded promoters drive expression through the gene encoded in the isolated DNA, and following splicing, produces a mature mRNA molecule. Using vectors encoding the appropriate reading frame in the activation exon, this process allows protein to be expressed from any gene encoded by the transfected genomic DNA.

To achieve stable expression of the activated gene, the transfected activation vector/genomic DNA can be integrated into the host cell genome. Alternatively, the transfected activation vector/genomic DNA can be maintained as a stable episome (e.g. using a viral origin of replication and/or nuclear retention function—see below). In yet another embodiment, the activated gene may be expressed transiently, for example, from a plasmid.

As used herein, the term “genomic DNA” refers to any DNA sequence derived from a genome. But the term also can apply to the unspliced genetic material from a cell. Splicing refers to the process of removing introns from genes following transcription. Thus, genomic DNA, in contrast to mRNA and cDNA, contains exons and introns in an unspliced form. In the present invention, genomic DNA derived from eukaryotic cells is particularly useful since most eukaryotic genes contain exons and introns, and since the vectors of the present invention are designed to express genes encoded in genomic DNA by splicing from the activation exons to the first downstream exon, thereby removing intervening introns.

Genomic DNA useful in the present invention can be isolated using any method known in the art. A number of methods for isolating high molecular weight genomic DNA and ultra-high molecular weight genomic DNA (intact and encased in agarose plugs) have been described (Sambrook et al., Molecular Cloning, Cold Spring Harbor Laboratory Press, (1989)). In addition, commercial kits for isolating genomic DNA of various sizes are also available (Gibco/BRL, Stratagene, ClonTech, etc.).

The genomic DNA used in the invention can encompass the entire genome of an organism. Alternatively, the genomic DNA may include only a portion of the entire genome from an organism. For example, the genomic DNA can contain multiple chromosomes, a single chromosome, a portion of a chromosome, a genetic locus, a single gene, or a portion of a gene.

Genomic DNA useful in the invention can be substantially intact (i.e., unfragmented) prior to introduction into a host cell. Alternatively, the genomic DNA can be fragmented prior to introduction into a host cell. This can be accomplished by, for example, mechanical shearing, nuclease treatment, chemical treatment, irradiation, or other methods known in the art. When the genomic DNA is fragmented, the fragmentation conditions can be adjusted to produce DNA fragments of any desirable size. Typically, DNA fragments should be large enough to contain at least one gene, or a portion of a gene (e.g. at least one exon).

The genomic DNA can be introduced directly into an appropriate eukaryotic host cell without prior cloning. Alternatively, the genomic DNA (or genomic DNA fragments) can be cloned into a vector prior to transfection. Useful vectors include, but are not limited to, high and intermediate copy number plasmids (e.g. pUC, pBluescript, pACYC184, pBR322, etc.), cosmids, bacterial artificial chromosomes (BACs), yeast artificial chromosomes (YACs), P1 artificial chromosomes (PACs), and phage (e.g. lambda, M13, etc.). Other cloning vectors known in the art can also be used. When genomic DNA has been cloned into a cloning vector, specific cloned DNA fragments can be isolated and used in the present invention. For example, YAC, BAC, PAC, or cosmid libraries can be screened by hybridization to identify clones that map to specific chromosomal regions. Optionally, once isolated, these clones can be ordered to produce a contig through the chromosomal region of interest. To rapidly isolate cDNA copies of the genes present in this contig, these genomic clones can be transfected, separately or en masse, with the activation vector into a host cell. cDNA containing a vector encoded exon, and lacking a vector encoded intron, can then be isolated and analyzed. Thus, since all genes present in a contig can be rapidly isolated as cDNA clones, this approach greatly enhances the speed of positional cloning approaches.

Any activation vector described herein, including derivatives recognized by those skilled in the art, can be co-transfected with genomic DNA, and therefore, are useful in the present invention. In its simplest form, the vector can one or more promoter/exon units. Examples of other useful vectors include, but are not limited to, poly A trap vectors, dual poly (A)/Splice acceptor trap vectors, bi-directional vectors, multi-promoter/activation exon vectors, vectors for isolating cDNAs corresponding to activated genes, and vectors for activating protein expression from activated.

The activation vector can also contain a viral origin of replication. The presence of a viral origin of replication allows vectors containing genomic fragments to be propagated as an episome in the host cell. Examples of useful viral origins of replication include ori P (Epstein Barr Virus), SV40 ori, BPV ori, and vaccinia ori. To facilitate replication from these origins, the appropriate viral replication proteins can be expressed from the vector. For example, EBV ori P and SV40 ori containing vectors can also encode and express EBNA-1 or T antigen, respectively. Alternatively, the vectors can be introduced into cells that are already expressing the viral replication protein (e.g. EBNA-1 or T antigen). Examples of cells expressing EBNA-1 and T antigen include human 293 cells transfected with an EBNA-1 expression unit (ClonTech) and COS-7 cells (American Type Culture Collection; ATCC No. CRL-1651), respectively.

The vector can also contain an amplifiable marker. This enables cells containing increased copies of the vector and flanking genomic DNA, either episomal or integrated in the host cell genome, to be isolated. Cells containing increased copies of the vector and flanking genomic DNA express the activated gene at higher levels, facilitating gene isolation and protein production.

The vector and genomic DNA can be introduced into any host cell capable of splicing from the vector-encoded splice donor site to a splice acceptor site encoded by the genomic DNA. In a preferred embodiment, the genomic DNA/activation vector are transfected into a host cell from the same species as the cell from which the genomic DNA was isolated. In some instances, however, it is advantageous to transfect the genomic DNA into a host cell from a species that is different from the cell from which the genomic DNA was isolated. For example, transfection of genomic DNA from one species into a host cell of a second species can facilitate analysis of the genes activated in the transfected genomic DNA using hybridization techniques. Under high stringency hybridization, activated genes that were encoded by the transfected DNA can be distinguished from genes derived from the host cell. Transfection of genomic DNA from one species into a host cell from another species can also be used to produce protein in a heterologous cell. This allows protein to be produced in heterologous cells that provide growth, protein modification, or manufacturing advantages.

The vector can be co-transfected into a host cell along with genomic DNA, wherein the vector is not attached to the genomic DNA prior to introduction into the cell. In this embodiment, the genomic DNA will become fragmented during the transfection process, thereby creating free DNA ends. These DNA ends can become joined to the co-transfected activation vector by the cell's DNA repair machinery. Following joining to the activation vector, the genomic DNA and activation vector can be integrated into the host cell genome by the process of non-homologous recombination. If, during this process, a vector becomes joined to a gene encoded by the transfected genomic DNA, the vector will activate its expression.

Alternatively, the non-targeted activation vector can be physically linked to the genomic DNA prior to transfection. In a preferred embodiment, genomic DNA fragments are ligated to the vector prior to transfection. This is advantageous because it maximizes the probability of the vector becoming operably linked to a gene encoded by the genomic DNA, and minimizes the probability of the vector integrating into the host cell genome without the heterologous genomic DNA.

In a related embodiment, the genomic DNA can be cloned into the activation vector, downstream of the activation exon. In this embodiment, cloning of large genomic fragments can be facilitated in vectors capable of accommodating large genomic fragments. Thus, the activation vector can be constructed in BACs, YACs, PACs, cosmids, or similar vectors capable of propagating large fragments of genomic DNA.

Another method for joining the activation vector to genomic DNA involves transposition. In this embodiment, the activation vector is integrated into the genomic DNA by transposition or retroviral integration reactions prior to transfection into a cell. Accordingly, activation vectors can contain cis sequences necessary for facilitating transposition and/or retroviral integration. Examples of vectors containing transposon signals are illustrated in FIG. 27; however, it is recognized that any vector described herein can contain transposon signals.

Any transposition system capable of inserting foreign sequences into genomic DNA can be used in the present invention. In addition, transposons capable of facilitating inversions and deletions can also be used to practice the invention. While deletion and inversion systems do not integrate the activation vector into genomic DNA, they do allow the activation vector to change positions relative to cloned genomic DNA when the genomic DNA has been cloned into the activation vector. Thus, multiple genes within a given genomic fragment can be activated by shuffling the activation vector (by integration, inversion, or deletion) into multiple positions within, or outside of, the genomic fragment. Examples of transposition systems useful for the present invention include, but are not limited to □□, Tn 3, Tn5, Tn7, Tn9, Tn10, Ty, retroviral integration and retro-transposons (Berg et al, Mobile DNA, ASM Press, Washington DC, pp. 879-925 (1989); Strathman et al.,(1991) Proc. Natl. Acad Sci. USA 88:1247; Berg et al. (1992) Gene 113:9; Liu et al. (1987) Nucl. Acids Res. 15:9461, Martin et al. (1995) Proc. Natl. Acad Sci. USA 92:8398; Phadnis et al. (1989) Proc. Natl. Acad Sci. USA 86:5908; Tomcsanyi et al (1990) J Bacteriol. 172:6348; Way et al. (1984) Gene 32:369; Bainton et al. (1991) Cell 65:805; Ahmed et al. (1984) J Mol. Biol. 178:941; Benjamin et al. (1989) Cell 59:373; Brown et al. (1987) Cell 49:347; Eichinger et al. (1988) Cell 54:955; Eichinger et al. (1990) Genes Dev. 4:324; Braiterman et al. (1994) Mol. Cell. Biol. 14:5719; Braiterman et al. (1994) Mol. Cell. Biol. 14:5731; York et al. (1998) Nucl. Acids Res. 26:1927; Devine et al. (1994) Nucl. Acids Res. 18:3765; Goryshin et al. (1998) J. Biol. Chem. 273:7367.

Using transposition, an activation vector can be integrated into any form of genomic DNA. For example, the activation vector can be integrated into either intact or fragmented genomic DNA. Alternatively, the activation vector can be integrated into a cloned fragment of genomic DNA (FIG. 28). In this embodiment, the genomic DNA can reside in any cloning vector, including high and intermediate copy number plasmids (e.g. pUC, pBluescript, pACYC184, pBR322, etc.), cosmids, bacterial artificial chromosomes (BACs), yeast artificial chromosomes (YACs), P1 artificial chromosomes (PACs), and phage (e.g. lambda, M13, etc.). Other cloning vectors known in the art can also be used. As described above, genomic fragments from specific genetic loci can be isolated an used as a substrate for activation vector integration.

Following integration of the activation vector, the genomic DNA can be introduced directly into a suitable host cell for expression of the activated gene. Alternatively, the genomic DNA can be introduced into and propagated in an intermediate host cell. For example, following integration of an activation vector into a BAC genomic library, the BAC library can be transformed into E. coli. This allows plasmids containing the transposon to be enriched by selecting for an antibiotic resistance marker residing on the activation vector. As a result, BAC plasmids lacking an integrated activation vector will be removed by antibiotic selection.

The transposition mediated activation vector integration can occur in vitro using purified enzymes. Alternatively, the transposition reaction can occur in vivo. For example, transposition can be carried out in bacteria, using a donor strain carrying the transposon either on a vector or as integrated copies in the genome. A target of interest is introduced into the transposer host where it receives integrations. Targets bearing insertions are then recovered from the host by genetic selection. Similarly, eukaryotic host cells, such as yeast, plant, insect, or mammalian cells, can be used to carry out the transposon mediated integration of an activation vector into a fragment of genomic DNA.

### References

1) Sambrook et al., Molecular Cloning, Cold Spring Harbor Press, New York (1989).

2) Treco et al., U.S. Pat. No. 5,541,670 (1997)

3) Treco et al., U.S. Pat. No. 5,733,761 (1998)

4) Treco et al., U.S. Pat. No. 5,968,502 (1999)

5) Skoultchi et al., U.S. Pat. No. 5,981,214 (1999)

6) Chappel, U.S. Pat. No. 5,272,071 (1993)

7) Harrington et aL, U.S. Pat. Application Ser. No. 09/276,820

8) Gluzman, (1982) Eukaryotic Viral Vectors, Cold Spring Harbor Laboratory Press, New York

9) Jalanko et al, (1988) Biochim Biophys Acta 949:206-212

10) Belt et al., (1989) Gene 84:407-417

11) DeBenedetti et al., (1991) Nucleic Acids Research 19:1925-1931

12) Kaufman, (1990) Methods in Enzymology 185:537-566

13) Natesan, U.S. Pat. No. 6,015,709 (2000)

14) Sangamo, WO 98/54311; WO 98/53057; U.S. Pat. No. 5,789,538; WO 96/20951

15) Mansour, (1988) Nature 336:348-352

16) Maccecchini, U.S. Pat. No. 5,854,217 (1998)

17) Kaczorowski et al., U.S. Pat. No. 5,637,470 (1997)

18) Cabot, U.S. Pat. No. 5,885,786 (1999)

19) Daggett et al, U.S. Pat. No. 5,807,689 (1998)

20) Brown et al., U.S. Pat. No. 5,962,314 (1999)

21) Thorens, U.S. Pat. No. 5,670,360 (1997)

22) Nemeth et al., U.S. Pat. No. 5,858,684 (1999)

23) Hartley, et al., U.S. Pat. No. 5,888,732 (1999)

24) Elledge, et al., U.S. Pat. No. 5,851,808 (1998)

25) Bebee, et al., U.S. Pat. No. 5,434,066 (1995)

26) Kolot et al., (1999) Mol. Biol. Rep. 26:207-213

27) Schlake et al, (1994) Biochemistry 33:12746-12751

28) Baubonis et al., (1993) Nucleic Acids Res. 11:2025-2029

That which is claimed:

1. A nucleic acid construct comprising at least two units, each unit comprising a promoter sequence operably linked to an exon and unpaired splice donor sequence, wherein at least two of said exons have a translation start codon in the same reading frame.

2. A nucleic acid construct comprising at least two units, each unit comprising a promoter sequence operably linked to an exon and unpaired splice donor sequence, wherein at least two of said exons lack a translation start site.

3. A nucleic acid construct comprising at least two units, each unit comprising a promoter sequence operably linked to an exon and unpaired splice donor sequence, each of said copies also being operably linked to a nucleic acid sequence X, wherein X is genomic DNA.

4. A nucleic acid construct comprising at least two units, each unit comprising a promoter sequence operably linked to an exon and unpaired splice donor sequence, each of said copies also being operably linked to a nucleic acid sequence X, wherein X:
(1) is a full length cDNA or part thereof;
(2) produces a ribozyme;
(3) produces antisense RNA; or
(4) is a synthetic sequence.

5. The nucleic acid construct of any of claims 1-4 further comprising one or more splice acceptor sequences operably linked to said splice donor sequence.

6. A vector containing any of the nucleic acid constructs of claims 1-4.

7. The vector of claim 6 wherein said vector is a retroviral vector.

8. The vector of claim 6 wherein said vector is a transposon vector.

9. The nucleic acid construct of any of claims 1-4 wherein said vector contains 5-10 of said copies.

10. The nucleic acid construct of any of claims 1-4 wherein said vector contains 10-15 of said copies.

11. The nucleic acid construct of any of claims 1-4 wherein said construct also contains a selectable marker.

12. The nucleic acid construct of any of claims 1-4 wherein said construct also contains an amplifiable marker.

13. The nucleic acid construct of any of claims 1-4 wherein said construct also contains site-specific recombination signals.

14. The nucleic acid construct of any of claims 1-4 wherein said construct also contains targeting sequences for homologous recombination.

15. A cell containing any of the vectors of claim 6.

16. A method of producing an expression product comprising culturing the nucleic acid construct of claim 3 or 4 in a cell wherein said nucleic acid sequence X is expressed, thus producing the expression product of said sequence X.

17. A method for producing an expression product, said method comprising introducing either of the nucleic acid constructs of claim 1 or claim 2 into a cell, allowing said constructs to recombine with a DNA sequence in said cell, wherein said DNA sequence is capable of producing an expression product, allowing said nucleic acid construct to recombine with said DNA sequence, culturing said cell to allow expression of said expression product, thus producing said expression product.


SEQUENCE LISTING (1 sequence)

SEQ ID NO: 1 - DNA, 8902 bp, Artificial Sequence
  Description of Artificial Sequence Synthetic DNA (pRIG-MP1)
agatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaatccaccctggctacaggtgagtactcggatctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaatccaccctggctacaggtgagtactcggatctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaatccaccctggctacaggtgagtactcggatctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggctctcttaaggtagcagatccttgctagagtcgaccaattctcatgtttgacagcttatcatcgcagatcctgagcttgtatggtgcactctcagtacaatctgctctgctgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgcccagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctgggtaccagctgctagcctcgagacgcgtgatttccttcgaagcttgtcatggttggttcgctaaactgcatcgtcgctgtgtcccagaacatgggcatcggcaagaacggggacctgccctggccaccgctcaggaatgaattcagatatttccagagaatgaccacaacctcttcagtagaaggtaaacagaatctggtgattatgggtaagaagacctggttctccattcctgagaagaatcgacctttaaagggtagaattaatttagttctcagcagagaactcaaggaacctccacaaggagctcattttctttccagaagtctagatgatgccttaaaacttactgaacaaccagaattagcaaataaagtagacatggtctggatagttggtggcagttctgtttataaggaagccatgaatcacccaggccatcttaaactatttgtgacaaggatcatgcaagactttgaaagtgacacgttttttccagaaattgatttggagaaatataaacttctgccagaatacccaggtgttctctctgatgtccaggaggagaaaggcattaagtacaaatttgaagtatatgagaagaatgattaatcgatcttaagtttaatctttcccgggggtaccgtcgactgcggccgcgaattccaagcttgagtattctatcgtgtcacctaaataacttggcgtaatcatggtcatatctgtttcctgtgtgaaattgttatccgctcacaattccacacaacatacgagccggaagcataaagtgtaaagcctggggtgcctaatgagtgagctaactcacattaattgcgttgcgcgatgcttccattttgtgagggttaatgcttcgagaagacatgataagatacattgatgagtttggacaaaccacaacaagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgattccggagcctgaatggcgaatggacgcgccctgtagcggcgcattaagcgcggcgggtgtggtggttacgcgcacgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcgcctgtgtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaagatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac